SEQUENCE LISTING (2 sequences)

SEQ ID NO: 1 - protein, 214 aa, Artificial Sequence
  Trastuzumab LC
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, Artificial Sequence
  Trastuzumab HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0102207 A1
(43) Pub. Date: Mar. 30, 2023

(54) ANTIBODY-DRUG CONJUGATES AND THEIR USE IN THERAPY

(71) Applicant: MC SAF, TOURS (FR)

(72) Inventors: Christine BALTUS, LA RICHE (FR); Ludovic JUEN, TOURS (FR)

(21) Appl. No.: 17/612,351

(22) PCT Filed: May 19, 2020
(86) PCT No.: PCT/FR2020/050833

§ 371 Date: Jul. 18, 2022

(30) Foreign Application Priority Data
May 20, 2019 (FR) 1905253

(51) Int. Cl. A61K 47/68; A61P 35/00; A61K 31/337; A61K 31/704; A61K 31/675; A61K 31/4196; A61K 39/395

(52) U.S. Cl. CPC A61K 47/6817; A61K 47/6855; A61P 35/00; A61K 31/337; A61K 31/704; A61K 31/675; A61K 31/4196; A61K 39/3955

(57) ABSTRACT

The invention relates to cytotoxic conjugates and antibody-drug conjugates, and to their use in therapy, in particular for treating HER2+ cancers.

### CROSS REFERENCE TO RELATED APPLICATIONS

This application is the National Phase of PCT International Application No. PCT/FR2020/050833, filed on May 19, 2020, which claims priority to French Patent Application No. 1905253, filed on May 20, 2019, the entire disclosures of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on 18 Nov. 2021, is named 0177_0185_sequence_listing.txt and is 6,069 bytes in size.

### TECHNICAL FIELD

The present invention relates to useful cytotoxic conjugates comprising an attachment head, a linker arm, a spacer and a cytotoxic agent.

The present invention also relates to novel antibody-drug conjugates comprising an antibody directed against the HER2 (human epidermal growth factor receptor 2) antigen coupled to a cytotoxic drug, and to the use thereof as drug, in particular in anti-cancer therapy.

### PRIOR ART

An antibody-drug conjugate (ADC) constitutes a means for the selective delivery of a cytotoxic drug. The antibody-drug conjugate thus makes it possible to combine the specificity of antibody targeting with novel and powerful effector functions via the agents with which they are conjugated.

The general structure of an antibody-drug conjugate is that of formula (II). The part linking the antibody and the drug is called the linker arm or linker. It can be grafted to the antibody via at least one of the eight cysteines that form the four inter-chain disulfide bridges. The number of cytotoxic drug molecules grafted to the antibody determines what is known as the Drug-to-Antibody Ratio (DAR).

After binding to its target antigen, the antibody is internalized in the cell by receptor-mediated endocytosis. The vesicles fuse with lysosomes in which the cytotoxic drug is released from the antibody via various mechanisms. The active cytotoxic drug then acts directly on the cell by inducing its death, and sometimes on neighboring cancerous cells via transport or diffusion into the environment. The antibody is therefore mainly used as a vector and delivers the cytotoxic drug into the cell.

Anti-HER2 antibodies have already been conjugated to cytotoxic drugs, including monomethyl auristatin E (MMAE). A cytotoxic conjugate bearing MMAE has been developed under the name “vedotin”. This cytotoxic drug has been used with various monoclonal antibodies for the preparation of antibody-drug conjugates. Mention may for example be made of brentuximab-vedotin or glembatumumab-vedotin.

MMAE has been conjugated to trastuzumab as described in the document by Bryant et al., Mol. Pharmaceutics, 2015, 12 (6), pp. 1872-1879). Nevertheless, the efficacy of the trastuzumab-MMAE described in this document is unsatisfactory, notably for hoping to effectively treat HER2+ cancers. This may be due to the low DAR.

There is therefore a need to develop anti-HER2-cytotoxic drug conjugates which are more effective and more stable.

### SUMMARY OF THE INVENTION

A first subject of the invention relates to a cytotoxic conjugate of formula (I) below:

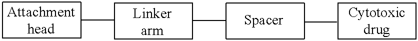

in which:
the attachment head is represented by either one of the two formulae below:

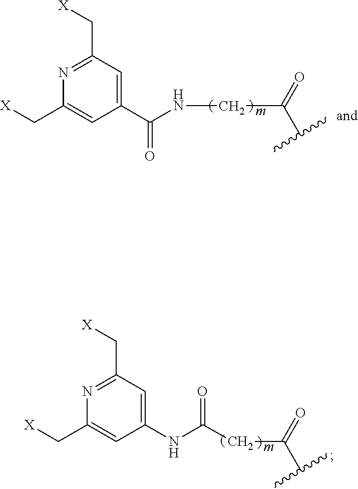

the linker arm is a cleavable linker arm chosen from the formulae below:

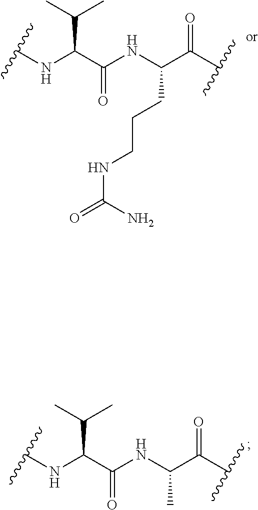 [SMILES: *N[C@H](C(=O)N[C@@H](CCCNC(N)=O)C(*)=O)C(C)C ; *N[C@H](C(=O)N[C@@H](C)C(*)=O)C(C)C]

the spacer is represented by the formula below:

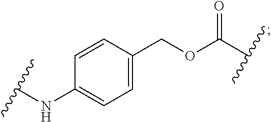 [SMILES: *Nc1ccc(COC(*)=O)cc1]

### X is Br, CI, I or F;

m is an integer ranging from 1 to 10, and preferably equal to 4 or 5.

A second subject of the invention relates to an antibody-drug conjugate of formula (II) below:

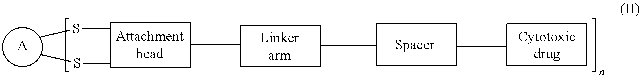

in which:
A is an anti-HER2 antibody or antibody fragment; the attachment head is represented by either one of the two formulae below:

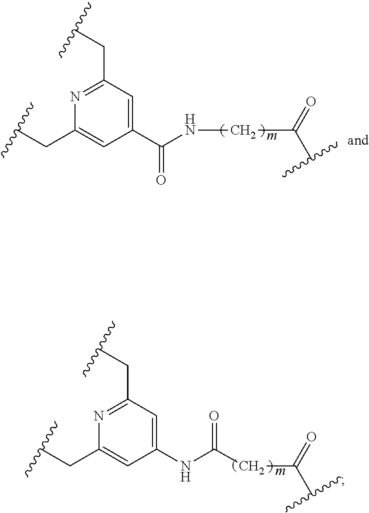 [SMILES: *Cc1cc(C(=O)NCC(*)=O)cc(C*)n1 ; *Cc1cc(NC(=O)CC(*)=O)cc(C*)n1]

the linker arm is a cleavable linker arm chosen from the formulae below:

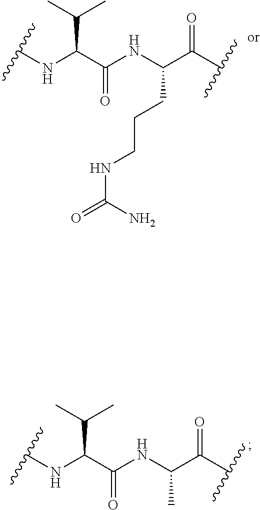 [SMILES: *N[C@H](C(=O)N[C@@H](CCCNC(N)=O)C(*)=O)C(C)C ; *N[C@H](C(=O)N[C@@H](C)C(*)=O)C(C)C]

the spacer is represented by the formula below:

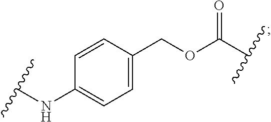 [SMILES: *Nc1ccc(COC(*)=O)cc1]

m is an integer ranging from 1 to 10, and preferably equal to 4 or 5;
n is an integer ranging from 1 to 4.

A third subject of the invention relates to a composition comprising one or more antibody-drug conjugate(s) according to the invention.

A fourth subject of the invention relates to an antibody-drug conjugate according to the invention or a composition according to the invention, for use as a medicament.

A fifth subject of the invention relates to an antibody-drug conjugate according to the invention or a composition according to the invention, for use in the treatment of an HER2+ cancer.

A sixth subject of the invention relates to a process for preparing a cytotoxic conjugate according to the invention, comprising a step which consists in coupling an attachment head of formula:

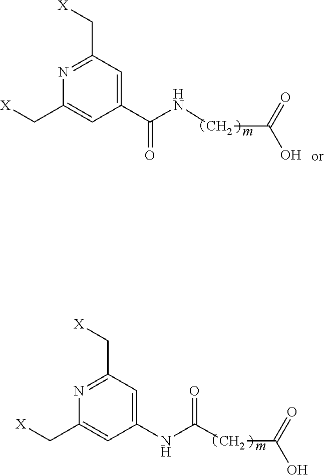

with a compound of formula

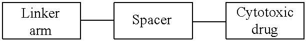

in which:
the linker arm is a cleavable linker arm chosen from the formulae below:

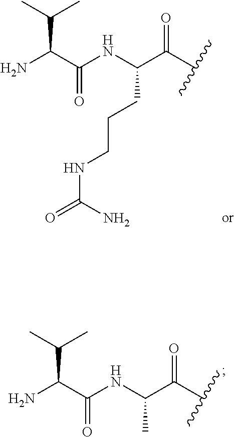 [SMILES: *C(=O)[C@H](CCCNC(N)=O)NC(=O)[C@@H](N)C(C)C ; *C(=O)[C@H](C)NC(=O)[C@@H](N)C(C)C]

the spacer is represented by the formula below:

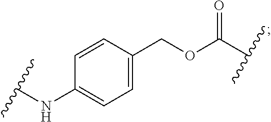 [SMILES: *Nc1ccc(COC(*)=O)cc1]

### X is Br, CI, I or F;

m is an integer ranging from 1 to 10, and preferably equal to 4 or 5.

Preferably, the process for preparing a cytotoxic conjugate according to the invention comprises a step which consists in coupling 6-(2,6-bis(bromomethyl)pyridin-4-yl)amidohexanoic acid or 6-((2,6-bis(bromomethyl)pyridin-4-yl)amino)-6-oxohexanoic acid with valine-citrulline-p-aminobenzylcarbamate-MMAE or a salt of said compound.

A seventh subject of the invention relates to a process for preparing an antibody-drug conjugate according to the invention, comprising the following steps:
(i) preparing a cytotoxic conjugate according to the process of the invention, and
(ii) reacting the cytotoxic conjugate obtained in step (i) with an anti-HER2 antibody or an anti-HER2 antibody fragment.

Preferably, the process for preparing an antibody-drug conjugate according to the invention comprises a step which consists in reacting 6-(2,6-bis(bromomethyl)pyridin-4-yl)amido-N-hexanamide-valine-citrulline-p-aminobenzylcarbamate-MMAE or 6-((2,6-bis(bromomethyl)pyridin-4-yl)amino)-6-oxohexanamide-valine-citrulline-p-aminobenzylcarbamate-MMAE with an anti-HER2 antibody or an anti-HER2 antibody fragment.

### DETAILED DESCRIPTION

Definitions

The term “cytotoxic conjugate” denotes a conjugate which comprises a cytotoxic drug.

The term “cytotoxic drug” denotes any natural or synthetic molecule capable of inhibiting or preventing cell function. The term “cytotoxic” is understood to mean the property, for a chemical or biological agent, of altering cells, possibly to the point of destroying them.

In a particular embodiment of the invention, the cytotoxic drug is chosen from any compound which has obtained a marketing authorization and which is used in anti-cancer or anti-inflammatory therapy, or any molecule undergoing clinical evaluation in terms of anti-cancer or anti-inflammatory therapy. The cytotoxic drug will be chosen, for example, from paclitaxel (Taxol®) or docetaxel (Taxotere®) or one of its derivatives, topotecan, bortezomib, daunorubicin, daunorubicin analogs, vincristine, mitomycin C, retinoic acid, methotrexate, Ilomedin, aspirin, IMiDs, lenalidomide, pomalidomide.

In another particular embodiment of the invention, the cytotoxic drug is selected from the group consisting of duocarmycin and its analogs, dolastatins, combretastatin and its analogs, calicheamicin, N-acetyl-y-calicheamicin (CMC), a derivative of calicheamicin, maytansine and its analogs, such as a derivative of the maytansinoid type, for example DM1 and DM4, auristatins and their derivatives, such as auristatin E, auristatin EB (AEB), auristatin EFP (AEFP), monomethyl auristatin E (MMAE), monomethyl auristatin F (MMAF), tubulysin, disorazole, epothilones, echinomycin, estramustine, cemadotin, eleutherobin, methopterin, actinomycin, mitomycin A, camptothecin, a derivative of camptothecin, SN38, TK1, amanitin, a pyrrolobenzodiazepine, a pyrrolobenzodiazepine dimer, a pyrrolopyridodiazepine, a pyrrolopyridodiazepine dimer, a DNA intercalator, a histone deacetylase inhibitor, or a tyrosine kinase inhibitor. In another particular embodiment of the invention, the drug M is selected from Pseudomonas exotoxin (PE), deBouganin, Bouganin, diphtheria toxin (DT) and ricin.

In a particular embodiment, the cytotoxic drug is chosen from methotrexate, IMiDs, duocarmycin, combretastatin, calicheamicin, monomethyl auristatin E (MMAE), monomethyl auristatin F (MMAF), maytansine, DM1, DM4, SN38, amanitin, pyrrolobenzodiazepine, pyrrolobenzodiazepine dimer, pyrrolopyridodiazepine, pyrrolopyridodiazepine dimer, a histone deacetylase inhibitor, a tyrosine kinase inhibitor, and ricin, preferably MMAE, represented by the formula below:

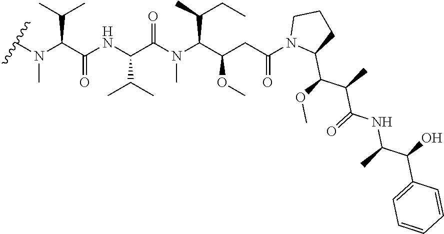 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

The term “antibody”, also referred to as “immunoglobulin” denotes a heterotetramer composed of two heavy chains of approximately 50-70 kDa each (called H chains for Heavy) and of two light chains of approximately 25 kDa each (called L chains for Light), linked together by intra-chain and inter-chain disulfide bridges. Each chain in the N-terminal position is composed of a variable region or domain, referred to as VL for the light chain and VH for the heavy chain, and in the C-terminal position is composed of a constant region consisting of a single domain referred to as CL for the light chain and of three or four domains referred to as CH1, CH2, CH3, CH4, for the heavy chain.

The term “chimeric antibody” is understood to mean an antibody of which the sequences of the variable regions of the light chains and of the heavy chains belong to a different species from that of the sequences of the constant regions of the light chains and of the heavy chains. For the purposes of the invention, the sequences of the variable regions of the heavy and light chains are preferentially of murine origin while the sequences of the constant regions of the heavy and light chains belong to a non-murine species. In this regard, for the constant regions, all species of non-murine mammals are able to be used, and in particular human, monkey, Suidae, Bovidae, Equidae, Felidae, Canidae or even birds, this list not being exhaustive. Preferably, the chimeric antibodies according to the invention contain sequences of constant regions of the heavy and light chains which are of human origin and sequences of variable regions of the heavy and light chains which are of murine origin.

The term “humanized antibody” is understood to mean an antibody of which all or some of the sequences of the regions involved in the recognition of the antigen (the hypervariable regions or CDRs: complementarity-determining regions) and sometimes certain amino acids of the FR regions (framework regions) are of non-human origin, while the sequences of the constant regions and variable regions not involved in recognition of the antigen are of human origin.

The term “human antibody” is understood to mean an antibody containing only human sequences, both for the variable and constant regions of the light chains and for the variable and constant regions of the heavy chains.

The term “antibody fragment” is understood to mean any part of an immunoglobulin obtained by enzymatic digestion or obtained by bioproduction and comprising at least one disulfide bridge, for example Fab, Fab′, F(ab′)₂, Fab′-SH, scFv-Fc or Fc.

The enzymatic digestion of immunoglobulins by papain generates two identical fragments, which are referred to as Fab (antigen-binding fragment) fragments, and an Fc fragment (crystallizable fragment). The enzymatic digestion of immunoglobulins by pepsin generates an F(ab′)₂ fragment and an Fc fragment split up into a plurality of peptides. F(ab′)₂ is formed of two Fab′ fragments linked by inter-chain disulfide bridges. The Fab parts are composed of the variable regions and the CH1 and CL domains. The Fab′ fragment is composed of the Fab region and a hinge region. Fab'-SH refers to a Fab′ fragment in which the cysteine residue of the hinge region bears a free thiol group. The scFv (single-chain variable fragment) is a fragment obtained from protein engineering which is composed solely of VH and VL variable domains. The structure is stabilized by a short, flexible peptide arm called a linker, which is placed between the two domains. The scFv fragment can be linked to an Fc fragment to form a scFv-Fc.

The term “HER2” denotes the “human epidermal growth factor receptor 2”, which is a membrane protein of the family of human epidermal growth factor receptors. “HER2” is also frequently referred to as “ErbB2”.

The term “HER2+ cancer” or “HER2-positive cancer” refers to a cancer involving amplified activation of HER2. In particular, the term “HER2+ cancer” denotes any case of cancer in which cancer cells exhibit deregulation of the HER2 gene. Preferably, in the context of the present invention, the HER2+ cancer is chosen from breast cancer, cancer of the female genital tract, such as endometrial cancer, uterine cancer or ovarian cancer, bladder cancer, anal cancer, colorectal cancer, in particular uterine papillary serous carcinoma, lung cancer, in particular non-small-cell lung cancer, liver cancer, kidney cancer, gastroesophageal cancer, stomach cancer, pancreatic cancer and gastric cancer. In a preferred embodiment, the HER2+ cancer is chosen from HER2+ breast cancer, HER2+ ovarian cancer, HER2+ bladder cancer, HER2+ colorectal cancer, HER2+ uterine papillary serous carcinoma, and an HER2+ gastric cancer, preferably HER2+ breast cancer.

The terms “purified” and “isolated” are understood to mean, with reference to an antibody according to the invention, that the antibody is present in the substantial absence of other biological macromolecules of the same type. The term “purified” as used herein preferably means at least 75% by weight, more preferably at least 85% by weight, even more preferably at least 95% by weight, and most preferably at least 98% by weight of antibody, relative to all of the macromolecules present.

The term “pharmaceutically acceptable” means approved by a federal or state regulatory agency or listed in the American or European Pharmacopeia or in another generally recognised pharmacopoeia, intended for use in animals and in humans. A “pharmaceutical composition” denotes a composition comprising a pharmaceutically acceptable vehicle. For example, a pharmaceutically acceptable vehicle may be a diluent, an adjuvant, an excipient or a vehicle with which the therapeutic agent is administered. These vehicles may be sterile liquids, such as water and oils, including those of petroleum, animal, plant or synthetic origin, such as peanut oil, soybean oil, mineral oil, sesame oil, etc. Water is a preferred vehicle when the pharmaceutical composition is administered intravenously. Saline solutions and aqueous solutions of dextrose and of glycerol may also be used as liquid vehicles, in particular for injectable solutions. Pharmaceutically acceptable excipients include starch, glucose, lactose, sucrose, sodium stearate, glycerol monostearate, talc, sodium chloride, skimmed milk powder, glycerol, propylene glycol, water, ethanol and the like. When the pharmaceutical composition is suitable for oral administration, the tablets or capsules may be prepared by conventional means using pharmaceutically acceptable excipients such as binders (for example pregelatinized corn starch, polyvinylpyrrolidone or hydroxypropyl methyl cellulose); fillers (for example lactose, microcrystalline cellulose or calcium hydrogen phosphate); lubricants (for example magnesium stearate, talc or silica); disintegrants (for example potato starch or sodium starch glycolate); or wetting agents (for example sodium lauryl sulfate). The tablets may be coated by processes that are well known in the prior art. Liquid preparations for oral administration may take the form for example of solutions, syrups or suspensions, or may be provided in the form of a dry product to be reconstituted with water or another appropriate vehicle prior to use. Such liquid preparations may be prepared by conventional means using pharmaceutically acceptable vehicles such as suspending agents (for example a sorbitol syrup, cellulose derivatives or hydrogenated edible fats); emulsifying agents (for example lecithin or acacia); nonaqueous vehicles (for example almond oil, oily esters, ethyl alcohol or fractionated vegetable oils); and preservatives (for example methyl or propyl p-hydroxybenzoates or sorbic acid). Pharmaceutical compositions may also contain buffer salts, flavorings, colorants and sweeteners, as appropriate. The composition according to the invention is preferably a pharmaceutical composition.

The term “treat” or “treatment” encompasses any beneficial or desirable effect on the symptoms of a pathology or of a pathological condition, and may even include a minimal reduction in one or more measurable markers of the pathology or of the pathological condition. Treatment may for example involve either reducing or improving the symptoms of the pathology or of the pathological condition, or delaying the progression of the disease or the pathological condition. The term “treatment” does not necessarily mean the complete eradication or curing of the pathology or the associated symptoms.

Cytotoxic conjugate

The invention relates to a cytotoxic conjugate of formula (I) below:

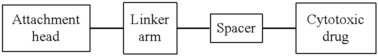

in which:
the attachment head is represented by either one of the two formulae below:

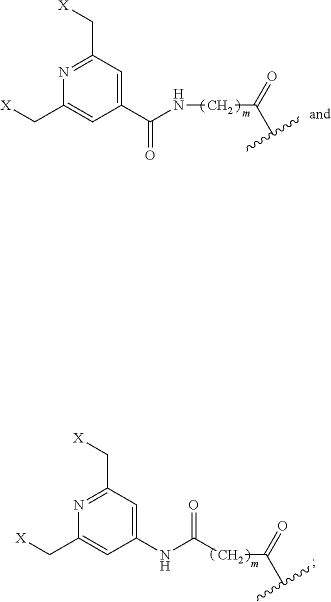

the linker arm is a cleavable linker arm chosen from the formulae below:

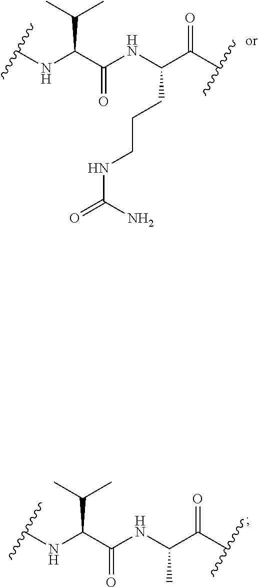 [SMILES: *N[C@H](C(=O)N[C@@H](CCCNC(N)=O)C(*)=O)C(C)C ; *N[C@H](C(=O)N[C@@H](C)C(*)=O)C(C)C]

the spacer is represented by the formula below:

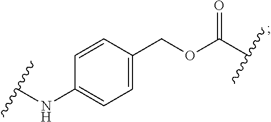 [SMILES: *Nc1ccc(COC(*)=O)cc1]

X is Br, CI, I or F, advantageously Br;
m is an integer ranging from 1 to 10, advantageously ranging from 2 to 7, from 3 to 6, and advantageously equal to 4 or 5.

In a particularly preferred embodiment, the cytotoxic conjugate corresponds to either one of the two formulae (la) below:

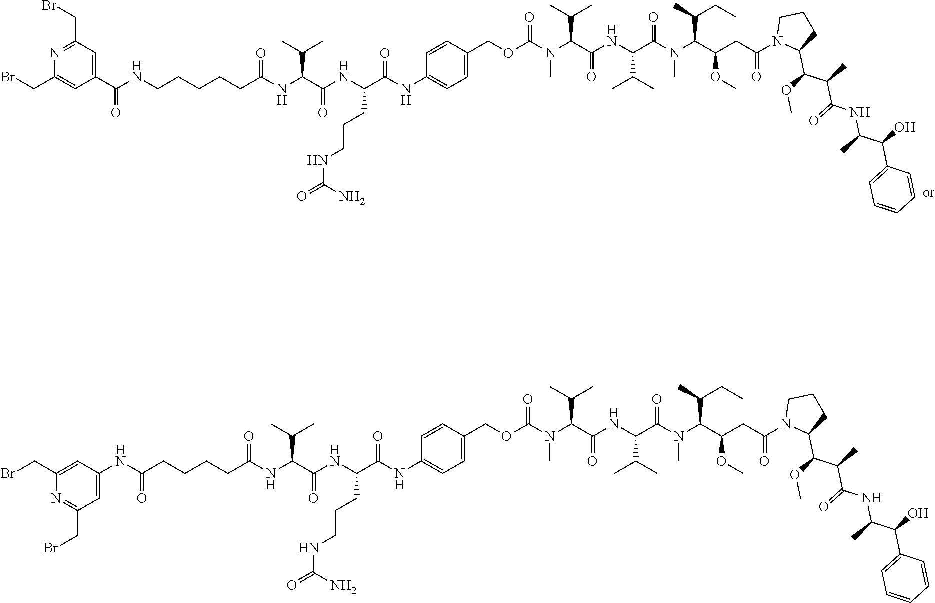 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCNC(=O)c2cc(CBr)nc(CBr)c2)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCC(=O)Nc2cc(CBr)nc(CBr)c2)C(C)C)cc1)C(C)C]

Antibody-drug conjugate

The invention also relates to an antibody-drug conjugate of formula (II) below:

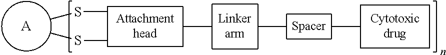

in which:
A is an anti-HER2 antibody or antibody fragment;
the attachment head is represented by either one of the two formulae below:

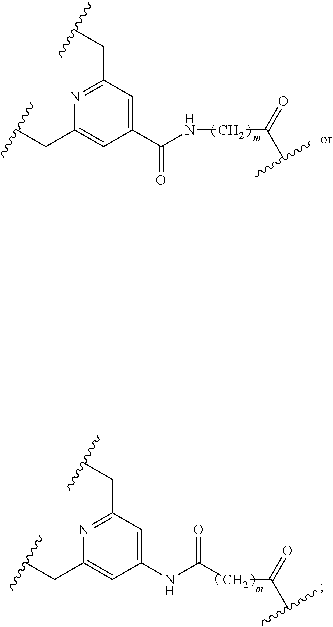 [SMILES: *Cc1cc(C(=O)NCC(*)=O)cc(C*)n1 ; *Cc1cc(NC(=O)CC(*)=O)cc(C*)n1]

the linker arm is a cleavable linker arm chosen from the formulae below:

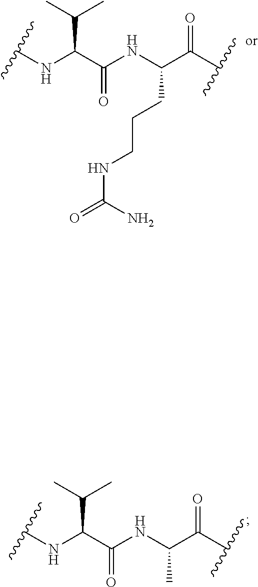 [SMILES: *N[C@H](C(=O)N[C@@H](CCCNC(N)=O)C(*)=O)C(C)C ; *N[C@H](C(=O)N[C@@H](C)C(*)=O)C(C)C]

the spacer is represented by the formula below:

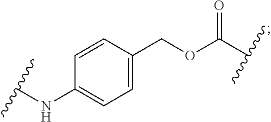 [SMILES: *Nc1ccc(COC(*)=O)cc1]

m is an integer ranging from 1 to 10, advantageously ranging from 2 to 7, from 3 to 6, and advantageously equal to 4 or 5;
n is an integer ranging from 1 to 4.

The anti-HER2 antibody or antibody fragment according to the invention may be of mammalian origin (for example human or mouse), humanized or chimeric. It is preferably a monoclonal antibody produced recombinantly by cells genetically modified according to techniques widely described in the prior art.

When A is an anti-HER2 antibody, it is preferably a human IgG, for example IgG1, IgG2, IgG3 or IgG4. In a particular embodiment, A is trastuzumab.

Trastuzumab is a humanized anti-HER2 antibody of IgG1 type having sequence SEQ ID NO: 1 for the light chain and SEQ ID NO: 2 for the heavy chain. Trastuzumab is notably marketed by the company Roche under the name Herceptin®.

In a particular embodiment, the antibody-drug conjugate of the invention has either one of the two formulae below:

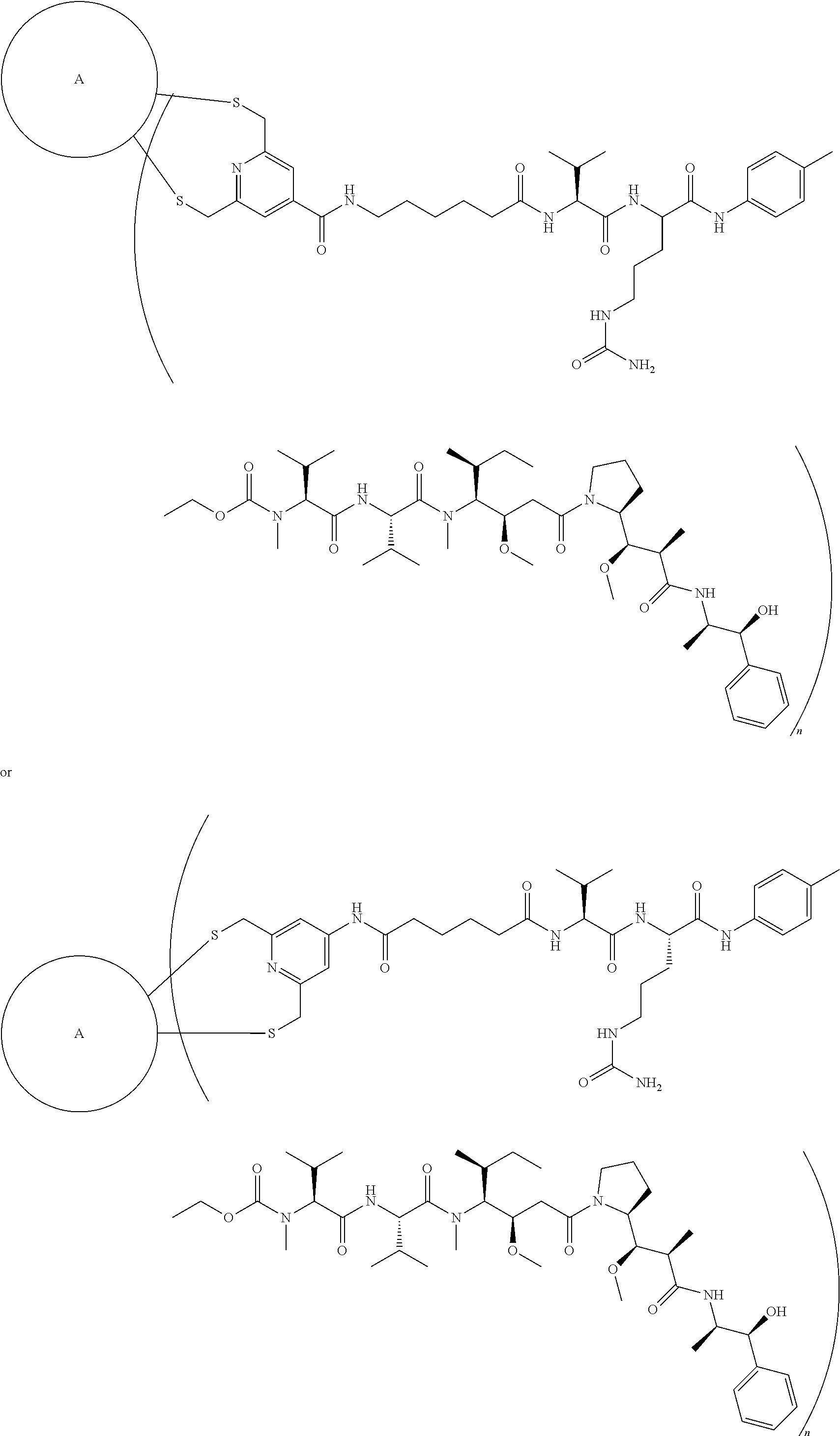

In a particular embodiment, the antibody-drug conjugate of the invention has either one of the two formulae (Ila) below:

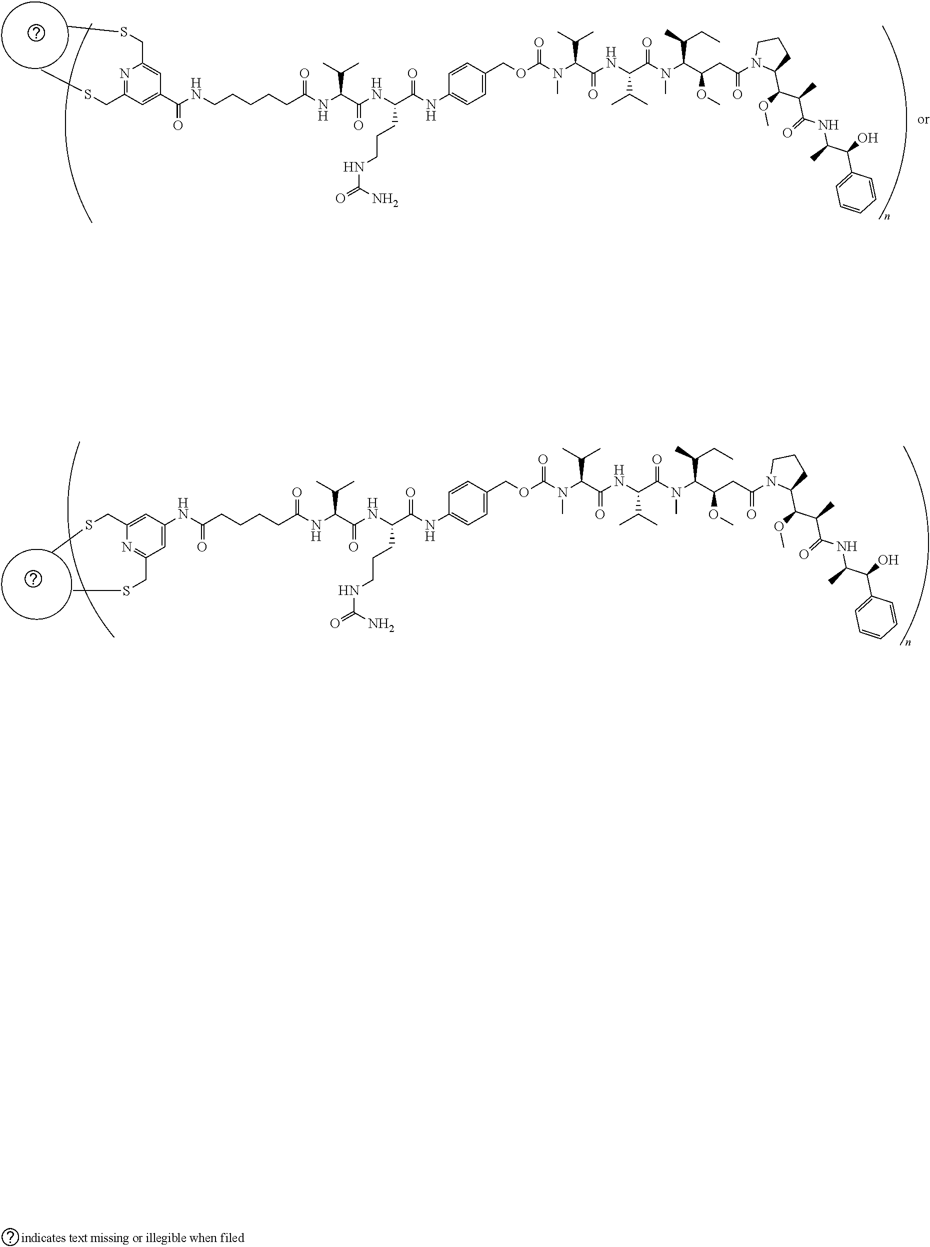

The antibody-drug conjugate of formula (Ila) is identified in the examples under the term “McSAF-pyridine” or “McSAF-pyridine retroamide”, respectively.

Advantageously, the antibody-drug conjugate according to the invention is purified (or isolated) using known purification techniques, such as purification on a chromatography and/or affinity column.

When n is equal to 1, the antibody-drug conjugate is commonly referred to as “DAR1”. When n is equal to 2, the antibody-drug conjugate is commonly referred to as “DAR2”. When n is equal to 3, the antibody-drug conjugate is commonly referred to as “DAR3”. When n is equal to 4, the antibody-drug conjugate is commonly referred to as “DAR4”.

In a particular embodiment, the antibody-drug conjugate has one or more effector functions mediated by the attenuated Fc part. Preferably, the effector function or functions mediated by the Fc part is/are chosen from ADCC (antibody-dependent cell-mediated cytotoxicity) and CDC (complement-dependent cytotoxicity). Those skilled in the art will have no difficulty with attenuating one or more effector functions mediated by the Fc part with regard to the teaching of the prior art, for example by mutating the Fc part. Many mutations are known for decreasing the effector functions mediated by the Fc part. It may for example be a mutation aiming to deglycosylate the Fc part, in particular to delete the glycosylation at the asparagine in position 297.

In a particular embodiment, the antibody-drug conjugate is deglycosylated at the Fc part, for example the antibody-drug conjugate no longer bears glycosylation at the asparagine in position 297.

Composition

The invention also relates to a composition comprising one or more antibody-drug conjugates of formula (II) as defined above. It may be a pharmaceutical composition comprising one or more antibody-drug conjugates of formula (II) as defined above and a pharmaceutically acceptable vehicle.

The composition according to the invention has the characteristic of being particularly homogeneous, which can result in better stability, better efficacy and/or a reduction in the side-effects of the composition, compared to a composition which is not homogeneous.

Advantageously, when A is an antibody, for example trastuzumab, the composition according to the invention is characterized by the following characteristics:
a) at least 50%, preferably at least 55%, at least 60%, at least 65%, for example at least 70%, at least 75%, at least 80%, at least 85%, at least 90%, at least 95%, at least 96%, at least 97%, at least 98% or at least 99%, of the antibody-drug conjugates of the composition have an n equal to 4;
b) the average Drug-to-Antibody Ratio (average DAR) is between 3.5 and 4.5, preferably between 3.8 and 4.2, between 3.9 and 4.1, between 3.9 and 4.0, for example equal to 4.0 ±0.2, 4.0 ±0.1, for example equal to 3.93 ±0.01. The average DAR is generally determined by the HIC (hydrophobic interaction chromatography) method or by native mass spectrometry. The HIC method and the native mass spectrometry method are widely described in the literature. It is possible to mention, for example, reference [1] for the HIC method and reference [2] for the native mass spectrometry method;
c) at least 95%, for example at least 96%, at least 97%, at least 98%, or at least 99%, of the antibody-drug conjugates are presence in the form of monomers. The monomer percentage is generally determined by the SEC (size exclusion chromatography) method. The SEC method is widely described in the literature, for example in reference [3];
d) one or more of the ratios of n defined below; or
e) a combination of two, three or four characteristics chosen from a), b), c) and d).

When A is an antibody, the composition according to the invention may include DAR0, that is to say antibodies without cytotoxic conjugate. Preferably less than 5%, less than 4%, less than 3%, less than 2%, less than 1%, less than 0.5%, less than 0.4%, less than 0.2%, or less than 0.1% DAR0, for example about 0% DAR0. The DAR0 percentage can be determined by the HIC (hydrophobic interaction chromatography) method or by native mass spectrometry.

The composition according to the invention may also include DARS, that is to say antibody-drug conjugates having 5 cytotoxic conjugates. Preferably less than 25%, less than 20%, less than 15%, less than 10%, less than 9%, less than 8%, less than 7%, less than 6%, or less than 5% DAR5. The presence of DAR5 in antibody-drug conjugate compositions is widely described in the literature, however the exact structure of the DAR5 has been little studied. Thus, the average DAR is calculated taking into account all the DARs present in the composition, that is to say the DAR0, the DAR1 (i.e. n=1), the DAR2 (i.e. n=2), the DAR3 (i.e. n=3), the DAR4 (i.e. n=4), the DAR5, etc. Preferably, when A is an antibody, the composition according to the invention comprises less than 1 DAR greater than DAR5, for example DAR6, DAR7, etc. Preferably, the composition according to the invention does not comprise any DAR greater than

DAR5, for example DAR6, DAR7, etc. The percentages of DAR5 and higher may be determined by the HIC (hydrophobic interaction chromatography) method or by native mass spectrometry.

When A is an antibody, for example trastuzumab, the composition according to the invention may be characterized by the following ratios of n:
less than 5%, less than 4%, less than 3%, less than 2%, less than 1, less than 0.75%, less than 0.5%, less than 0.25%, less than 0.1%, for example around 0.1% or around 0%, of the antibody-drug conjugates of the composition have an n equal to 1,
less than 5%, less than 4%, less than 3%, less than 2%, less than 1, less than 0.75%, less than 0.5%, less than 0.25%, less than 0.1%, for example around 0.5% or around 0%, of the antibody-drug conjugates of the composition have an n equal to 2,
less than 20%, less than 15%, less than 10%, less than 5%, less than 4%, less than 3%, less than 2%, less than 1%, for example around 8% or around 10%, of the antibody-drug conjugates of the composition have an n equal to 3, and
at least 50%, at least 55%, at least 60%, at least 65%, for example at least 70%, at least 75%, at least 80%, at least 85%, at least 90%, at least 95%, at least 96%, at least 97%, at least 98% or at least 99%, for example around 80% or around 70% ±5%, of the antibody-drug conjugates of the composition have an n equal to 4.

When A is an antibody, for example trastuzumab, the composition according to the invention may also be characterized by the following ratios of n:
between 0% and 5%, between 0% and 2%, between 0% and 1%, between 0% and 0.75%, between 0% and 0.5%, between 0% and 0.25%, between 0% and 0.1%, of the antibody-drug conjugates of the composition have an n equal to 1, between 0% and 5%, between 0% and 2%, between 0% and 1%, between 0% and 0.75%, between 0% and 0.5%, between 0% and 0.25%, between 0% and 0.1%, of the antibody-drug conjugates of the composition have an n equal to 2,
between 0% and 5%, between 5% and 15%, between 8% and 12%, between 8% and 9%, between 9% and 11%, of the antibody-drug conjugates of the composition have an n equal to 3, and
between 50% and 75%, between 65% and 70%, between 70% and 85%, between 75% and 80%, for example at least 70%, at least 75%, at least 80%, at least 85%, at least 90%, at least 95%, at least 96%, at least 97%, at least 98% or at least 99%, of the antibody-drug conjugates of the composition have an n equal to 4.

The ratios of n can be determined by the HIC (hydrophobic interaction chromatography) method or by native mass spectrometry.

In a first particular embodiment, when A is an antibody, for example trastuzumab, the composition according to the invention may be characterized by the following ratios of n determined by the HIC (hydrophobic interaction chromatography) method:
less than 5%, less than 4%, less than 3%, less than 2%, less than 1%, less than 0.75%, less than 0.5%, less than 0.25%, for example around 0.1%, of the antibody-drug conjugates of the composition have an n equal to 1,
less than 5%, less than 4%, less than 3%, less than 2%, less than 1%, less than 0.75%, for example around 0.5%, of the antibody-drug conjugates of the composition have an n equal to 2,
less than 20%, less than 15%, less than 10%, less than 5%, less than 4%, less than 3%, less than 2%, less than 1%, for example around 10%, of the antibody-drug conjugates of the composition have an n equal to 3, and/or
at least 50%, at least 55%, at least 60%, at least 65%, for example at least 70%, at least 75%, at least 80%, at least 85%, at least 90%, at least 95%, at least 96%, at least 97%, at least 98% or at least 99%, for example around 70% ±5%, of the antibody-drug conjugates of the composition have an n equal to 4.

Thus, when A is an antibody, for example trastuzumab, the composition according to the invention may also be characterized by the following ratios of n determined by the HIC (hydrophobic interaction chromatography) method:
between 0% and 5%, between 0% and 2%, between 0% and 1%, between 0% and 0.75%, between 0% and 0.5%, between 0% and 0.25%, of the antibody-drug conjugates of the composition have an n equal to 1,
between 0% and 5%, between 0% and 2%, between 0% and 1%, between 0% and 0.75%, of the antibody-drug conjugates of the composition have an n equal to 2,
between 0% and 5%, between 5% and 15%, between 8% and 12%, between 9% and 11%, of the antibody-drug conjugates of the composition have an n equal to 3, and
between 50% and 75%, between 65% and 70%, between 70% and 85%, between 75% and 80%, for example at least 70%, at least 75%, at least 80%, at least 85%, at least 90%, at least 95%, at least 96%, at least 97%, at least 98% or at least 99%, of the antibody-drug conjugates of the composition have an n equal to 4.

In a second particular embodiment, when A is an antibody, for example trastuzumab, the composition according to the invention may be characterized by the following ratios of n determined by native mass spectrometry:
less than 5%, less than 4%, less than 3%, less than 2%, less than 1%, less than 0.75%, less than 0.5%, less than 0.25%, less than 0.1%, for example around 0.1% or around 0%, of the antibody-drug conjugates of the composition have an n equal to 1,
less than 5%, less than 4%, less than 3%, less than 2%, less than 1%, less than 0.75%, less than 0.5%, less than 0.25%, less than 0.1%, for example around 0.5% or around 0%, of the antibody-drug conjugates of the composition have an n equal to 2,
less than 20%, less than 15%, less than 10%, less than 5%, less than 4%, less than 3%, less than 2%, less than 1%, for example around 8%, of the antibody-drug conjugates of the composition have an n equal to 3, and/or
at least 50%, at least 55%, at least 60%, at least 65%, for example at least 70%, at least 75%, at least 80%, at least 85%, at least 90%, at least 95%, at least 96%, at least 97%, at least 98% or at least 99%, for example around 80%, of the antibody-drug conjugates of the composition have an n equal to 4.

Thus, when A is an antibody, for example trastuzumab, the composition according to the invention may also be characterized by the following ratios of n determined by native mass spectrometry:
between 0% and 5%, between 0% and 2%, between 0% and 1%, between 0% and 0.75%, between 0% and 0.5%, between 0% and 0.25%, between 0% and 0.1%, of the antibody-drug conjugates of the composition have an n equal to 1,
between 0% and 5%, between 0% and 2%, between 0% and 1%, between 0% and 0.75%, between 0% and 0.5%, between 0% and 0.25%, between 0% and 0.1%, of the antibody-drug conjugates of the composition have an n equal to 2,
between 0% and 5%, between 5% and 15%, between 8% and 12%, between 8% and 9%, of the antibody-drug conjugates of the composition have an n equal to 3, and
between 50% and 75%, between 65% and 70%, between 70% and 85%, between 75% and 80%, for example at least 70%, at least 75%, at least 80%, at least 85%, at least 90%, at least 95%, at least 96%, at least 97%, at least 98% or at least 99%, of the antibody-drug conjugates of the composition have an n equal to 4.

In a very particular embodiment, the composition according to the invention has the HIC profile of FIG. 1 or the HIC profile of FIG. 19, or the native mass spectrometry profile of FIG. 5 or the native mass spectrometry profile of FIG. 21.

Therapeutic use

The invention also relates to an antibody-drug conjugate of formula (II) according to the invention or to a composition comprising an antibody-drug conjugate of formula (II) according to the invention for use as medicament, for example for use in the treatment of an HER2+ cancer, such as HER2+ breast cancer.

The antibody-drug conjugate or the composition according to the invention is preferably formulated for parenteral administration, for example intravascular (intravenous or intra-arterial), intraperitoneal or intramuscular administration. The term “parenterally administered”, as used herein, denotes modes of administration other than enteral and topical administration, generally by injection, and includes, without limitation, intravascular, intravenous, intramuscular, intra-arterial, intrathapsal, intracapsular, intra-orbital, intra-tumoral, intracardiac, intradermal and intraperitoneal administration, administration by injection, administration by transtracheal perfusion, and subcutaneous, intra-articular, subcapsular, subarachnoid, intraspinal and intrasternal administration. Preference within the context of the present invention is given to intravenous administration, for example via intravenous perfusion.

The dose of antibody-drug conjugate administered to a subject in need thereof will vary depending on a number of factors including, without limitation, the administration route, the type and the severity of the pathology being treated, the condition of the patient, the size of the patient, the age of the patient etc. Those skilled in the art can easily determine, based on their knowledge in this field, the dosage range required in accordance with these and other factors. The appropriate dose may also be determined using animal models or clinical trials. For example, typical doses of antibody-drug conjugate may be 1 mg/kg, 2 mg/kg, 3 mg/kg, 5 mg/kg, 6 mg/kg, 7 mg/kg, 8 mg/kg or more. The administration may be performed in a single dose or, more generally, in several doses. The administration schedule may comprise an initial loading dose followed by maintenance doses, for example weekly, every 2 weeks, every three weeks, every month, or more. The duration of treatment may vary depending on the pathology being treated and on the subject.

The antibody-drug conjugate or the composition according to the invention may be used in monotherapy or in combination with drugs having a recognized therapeutic benefit in the pathology under consideration. These may include, for example, paclitaxel, docetaxel, doxorubicin, cyclophosphamide, an aromatase inhibitor such as anastrozole, or an antibody used in anti-cancer immunotherapy such as an anti-PD1 antibody.

The description also relates to a method for treating an HER2+ cancer in a subject, comprising the administration to the subject of a therapeutically effective amount of an antibody-drug conjugate of formula (II) according to the invention or of a composition comprising an antibody-drug conjugate of formula (II) according to the invention.

Preparation process

The description also relates to a process for preparing a cytotoxic conjugate according to the invention.

The description also relates to a process for preparing antibody-drug of formula (II) as defined above, in which a cytotoxic conjugate of formula (I) as defined above is reacted with an anti-HER2 antibody or antibody fragment.

Another subject of the invention relates to a process for preparing a cytotoxic conjugate according to the invention, comprising a step which consists in coupling an attachment head of formula:

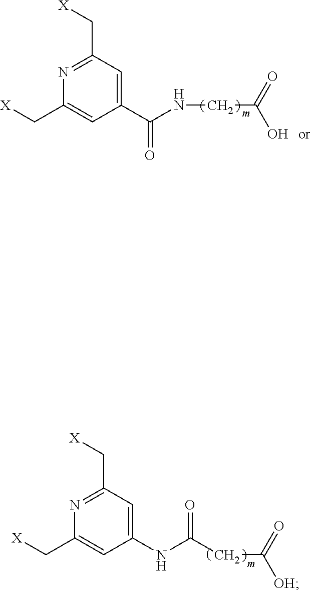

with a compound of formula

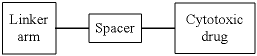

in which:
the linker arm is a cleavable linker arm chosen from the formulae below:

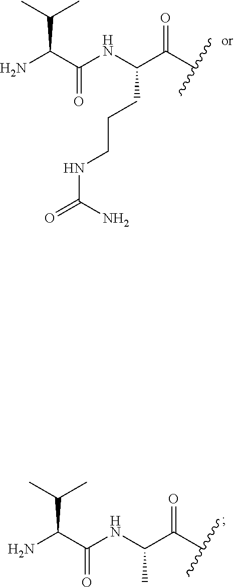 [SMILES: *C(=O)[C@H](CCCNC(N)=O)NC(=O)[C@@H](N)C(C)C ; *C(=O)[C@H](C)NC(=O)[C@@H](N)C(C)C]

the spacer is represented by the formula below:

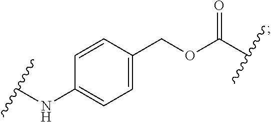 [SMILES: *Nc1ccc(COC(*)=O)cc1]

### X is Br, CI, I or F;

m is an integer ranging from 1 to 10, and preferably equal to 4 or 5.

The attachment head is described in more detail in the “cytotoxic conjugate” and “antibody-drug conjugate” sections hereinabove, with the difference that the attachment head used in the process comprises a terminal carboxylic acid function.

The linker arm is described in more detail in the “cytotoxic conjugate” and “antibody-drug conjugate” sections hereinabove, with the difference that the linker arm used in the process comprises a terminal amine function.

The spacer is described in more detail in the “cytotoxic conjugate” and “antibody-drug conjugate” sections hereinabove.

The cytotoxic drug is described in more detail in the “cytotoxic conjugate” and “antibody-drug conjugate” sections hereinabove.

In a particular embodiment, the process for preparing a cytotoxic conjugate according to the invention comprises a step which consists in coupling 6-(2,6-bis(bromomethyl)pyridin-4-yl)amidohexanoic acid (reference (7) in example 1A) or 6-((2,6-bis(bromomethyl)pyridin-4-yl)amino)-6-oxohexanoic acid (reference (16) in example 1B) with valine-citrulline-p-aminobenzylcarbamate-MMAE or a salt of said compound. In this particular embodiment, the process for preparing a cytotoxic conjugate according to the invention makes it possible to obtain 6-(2,6-bis(bromomethyl)pyridin-4-yl)amido-N-hexanamide-valine-citrulline-p-aminobenzylcarbamate-MMAE (reference (8) in example 1A) or 6-((2,6-bis(bromomethyl)pyridin-4-yl)amino)-6-oxohexanamide-valine-citrulline-p-aminobenzylcarbamate-MMAE (reference (17) in example 1B), respectively.

Another subject of the invention relates to a process for preparing an antibody-drug conjugate according to the invention, comprising the following 0.steps:
(i) preparing a cytotoxic conjugate according to the process of the invention, and
(ii) reacting the cytotoxic conjugate obtained in step (i) with an anti-HER2 antibody or an anti-HER2 antibody fragment.

The anti-HER2 antibody is described in more detail in the “cytotoxic conjugate” and “antibody-drug conjugate” sections hereinabove.

In a particular embodiment, the process for preparing an antibody-drug conjugate according to the invention comprises a step which consists in reacting 6-(2,6-bis(bromomethyl)pyridin-4-yl)amido-N-hexanamide-valine-citrulline-p-aminobenzylcarbamate-MMAE (reference (8) in example 1A) or 6-((2,6-bis(bromomethyl)pyridin-4-yl)amino)-6-oxohexanamide-valine-citrulline-p-aminobenzylcarbamate-MMAE (reference (17) in example 1B) with an anti-HER2 antibody or an anti-HER2 antibody fragment.

### BRIEF DESCRIPTION OF THE FIGURES

FIG. 1 represents the HIC (hydrophobic interaction chromatography) profile of a McSAF-pyridine antibody-drug conjugate composition according to the invention. The figure shows that the composition is enriched in DAR4, with around 69% DAR4.

FIG. 2 represents an SEC (size exclusion chromatography) analysis of a McSAF-pyridine antibody-drug conjugate composition according to the invention.

The figure shows that the composition is extremely homogeneous, with more than 99% monomer.

FIG. 3 represents the distribution of the DARs of 14 independent bioconjugations on the 1 mg scale. This demonstrates the high reproducibility of the bioconjugation for obtaining the McSAF-pyridine antibody-drug conjugate.

FIG. 4 represents the distribution of the DARs of various bioconjugations on various scales (1 mg, 2.5 mg and 5 mg scales). This demonstrates the high reproducibility of the bioconjugation for obtaining the McSAF-pyridine antibody-drug conjugate irrespective of the scale.

FIG. 5 represents the distribution of the DARs of a representative bioconjugation for obtaining the McSAF-pyridine antibody-drug conjugate by native mass spectrometry analysis. The figure demonstrates the chemical nature of the conjugate and shows that the composition is enriched in DAR4, with around 80% DAR4.

FIG. 6 represents the recognition of the HER2 antigen by McSAF-pyridine, T-DM1 and trastuzumab.

FIG. 7 represents the in vitro cytotoxicity of McSAF-pyridine on cells expressing HER2, or cells not expressing HER2, compared to T-DM1 and MMAE alone.

FIG. 8 represents the amount of MMAE released in human plasma by LC-MS/MS, comparing McSAF-pyridine with an ADC prepared using maleimide coupling chemistry (McSAF-maleimide1). This figure reflects the stability of the McSAF-pyridine antibody-drug conjugate.

FIG. 9 represents the change in the average DAR in solution at 37° C. of McSAF-pyridine compared to an ADC prepared using maleimide coupling chemistry (McSAF-maleimide1). This figure reflects the stability of the McSAF-pyridine antibody-drug conjugate.

FIG. 10 represents the change in the average DAR in solution in the presence of HSA (human serum albumin) at 37° C. of McSAF-pyridine compared to an ADC prepared using maleimide coupling chemistry (McSAF-maleimide1). This figure reflects the stability of the McSAF-pyridine antibody-drug conjugate.

FIG. 11 represents the change in the average DAR in solution at 40° C. over 28 days of McSAF-pyridine compared to an ADC prepared using maleimide coupling chemistry (McSAF-maleimide1), as measured by the HIC method. This figure reflects the stability of the McSAF-pyridine antibody-drug conjugate.

FIG. 12 represents the change in the DAR4 percentage in solution at 40° C. over 28 days of McSAF-pyridine compared to an ADC prepared using maleimide coupling chemistry (McSAF-maleimide1), as measured by the HIC method. This figure reflects the stability of the McSAF-pyridine antibody-drug conjugate.

FIG. 13 represents the change in the monomer percentage in solution at 40° C. over 28 days of McSAF-pyridine compared to an ADC prepared using maleimide coupling chemistry (McSAF-maleimide1), as measured by the SEC method.

FIG. 14 represents the change in tumor volume of mice treated with 5 mg/kg of McSAF-pyridine, 5 mg/kg of T-DM1 or a vehicle without ADC.

FIG. 15 represents the distribution of the tumor volumes of all of the mice treated with 5 mg/kg of McSAF-pyridine, 5 mg/kg of T-DM1 or a vehicle without ADC at day 70. This figure reflects the homogeneity of the anti-tumor response to the antibody-drug conjugate at 5 mg/kg.

FIG. 16 represents the change in tumor volume of mice treated with 1 mg/kg of McSAF-pyridine, 1 mg/kg of T-DM1 or a vehicle without ADC.

FIG. 17 represents the distribution of the tumor volumes of all of the mice treated with 1 mg/kg of McSAF-pyridine, 1 mg/kg of T-DM1 or a vehicle without ADC at day 70. This figure reflects the different anti-tumor responses to the antibody-drug conjugate and to T-MD1 at 1 mg/kg.

FIG. 18 represents the denaturing mass spectrometry profile of the McSAF-pyridine conjugate. The figure shows the presence of a completely reconstructed and conjugated species (LHHL DAR4).

FIG. 19 represents the HIC (hydrophobic interaction chromatography) profile of a McSAF-pyridine retroamide antibody-drug conjugate composition according to the invention. The figure shows that the composition is enriched in DAR4, with around 68% DAR4.

FIG. 20 represents the denaturing mass spectrometry profile of the McSAF-pyridine retroamide conjugate. The figure shows the presence of a completely reconstructed and conjugated species (LHHL DAR4).

FIG. 21 represents the distribution of the DARs of a representative bioconjugation for obtaining the McSAF-pyridine retroamide antibody-drug conjugate by native mass spectrometry analysis. The figure demonstrates the chemical nature of the conjugate and shows that the composition is enriched in DAR4, with around 75% DAR4.

FIG. 22 represents the distribution of the DARs of 5 independent bioconjugations on the 250 pg scale. This demonstrates the high reproducibility of the bioconjugation for obtaining the McSAF-pyridine retroamide antibody-drug conjugate.

### EXAMPLE

Example 1A: Synthesis of a Cytotoxic Conjugate According to the Invention (Pyridine)

General reaction scheme

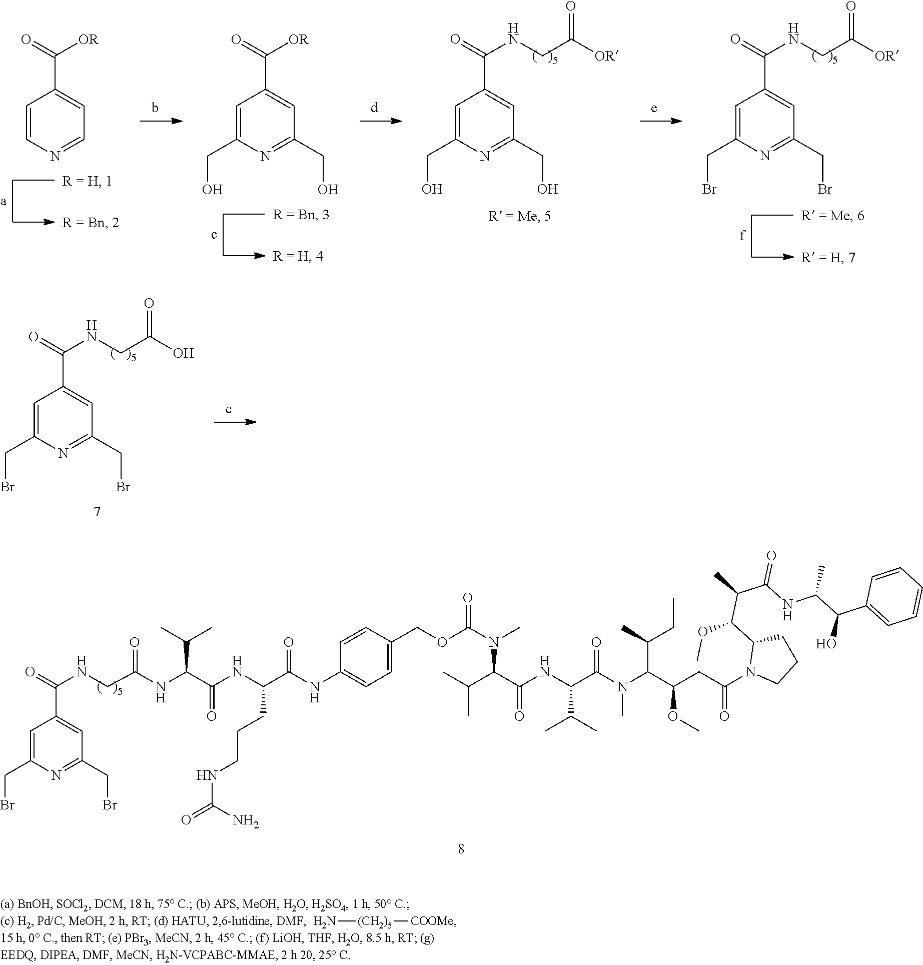

Detailed Reaction Scheme

Preparation of benzyl isonicotinate (2)

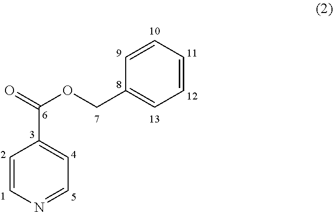 [SMILES: O=C(OCc1ccccc1)c1ccncc1]

Isonicotinic acid (1) (5.00 g; 40.614 mmol; 1.0 eq) was solubilized in thionyl chloride (15 mL; 206.77 mmol; 5.1 eq) and refluxed overnight. After returning to room temperature, the excess thionyl chloride was removed by evaporation under reduced pressure and then the residue obtained was dissolved in anhydrous dichloromethane (55 mL). Benzyl alcohol was added (4.2 mL; 40.614 mmol; 1.0 eq) and the mixture was stirred at reflux for 10 h. After returning to room temperature, the reaction medium was neutralized with a saturated sodium hydrogen carbonate solution and extracted with dichloromethane (3×100 mL). The organic phases were combined, washed with a saturated sodium chloride solution, dried over magnesium sulfate and concentrated under reduced pressure. The product obtained was purified by flash chromatography (SiO₂, cyclohexane/ethyl acetate 50:50) to give (2) (6.97 g; 80%) in the form of a colorless oil.

¹H NMR (300 MHz, DMSO) δ 8.80 (dd; J=6.1; 1.6 Hz; 2H_{1,5}), 7.86 (dd; J=6.1; 1.6 Hz, 2H_{2,4}), 7.56-7.29 (m; 5H₉₋₁₃), 5.39 (s; 2H₇).

¹³C NMR (75 MHz, DMSO) δ 165.0 (1C₆); 151.3 (2C_{1,5}); 137.2 (1C₃); 136.1 (1C8); 129.0 (2C_{10, 12}); 128.8 (1C₁₁); 128.6 (2C_{9,13}); 123.0 (2C_{2,4}); 67.4 (1C₇).

HRMS (ESI): neutral mass calculated for C₁₃H₁₁NO₂ [M]: 213.0790; observed 213.0796.

Preparation of benzyl 2,6-bis(hydroxymethyl)isonicotinate (3)

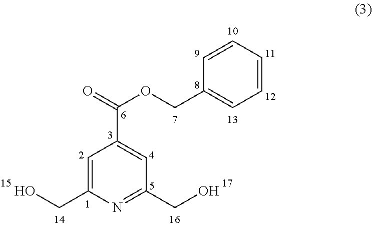 [SMILES: O=C(OCc1ccccc1)c1cc(CO)nc(CO)c1]

Benzyl isonicotinate (2) (2.48 g; 11.630 mmol; 1.0 eq) was dissolved in methanol (43 mL), stirred at 50° C. and concentrated sulfuric acid (320 μL; 6.016 mmol; 0.52 eq) was added. A solution of ammonium persulfate (26.500 g; 116.000 mmol; 10.0 eq) in water (43 mL) was added in two steps: a first rapid addition of 30 drops, a white suspension forming, then dropwise rapidly for 5 min. The reaction ramps up to 75° C., and then the yellow solution obtained was stirred at 50° C. for an additional 1 h. After returning to room temperature, the methanol was evaporated under reduced pressure. 50 mL of ethyl acetate were added and the medium was neutralized by addition of a saturated sodium hydrogen carbonate solution. The aqueous phase was extracted with ethyl acetate (3x100 mL) and the combined organic phases were washed with a saturated sodium chloride solution, dried over magnesium sulfate, and then concentrated under reduced pressure. The crude product was purified by flash chromatography

(Si02, dichloromethane/methanol, 95:5) to give (3) (1.56 g; 49%) in the form of a beige solid.

¹H NMR (300 MHz, DMSO) δ 7.81 (s; 2H_{2,4}); 7.55-7.32 (m; 5H₉₋₁₃); 5.60 (t; J=5.9 Hz; 2H_{15,17}); 5.40 (s; 2H₇); 4.59 (d; J=5.9 Hz; 4H_{14,16}).

¹³C NMR (75 MHz, DMSO) δ 165.0 (1C₆); 162.8 (2C_{1,5}); 138.0 (1C₃); 135.7 (1C₈); 128.6 (2C_{10, 12}); 128.4 (1C₁₁); 128.3 (2C_{9,13}); 117.0 (2C_{2,4}); 66.9 (1C₇); 63.9(2C_{14,16}).

HRMS (ESI): neutral mass calculated for C₁₅H₁₅NO₄ [M]: 273.1001; observed 273.1001.

Preparation of 2,6-bis(hydroxymethyl)isonicotinic acid (4)

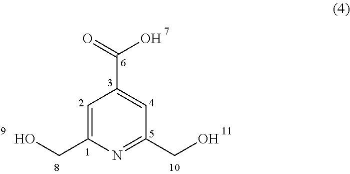 [SMILES: O=C(O)c1cc(CO)nc(CO)c1]

Benzyl 2,6-bis(hydroxymethyl)isonicotinate (3) (1.33 g; 4.867 mmol; 1.0 eq) was dissolved in methanol (50 mL) and the solution was degassed with argon for 15 min. 10% by weight palladium-on-charcoal (133 mg) was added and the reaction medium was stirred at room temperature under a hydrogen atmosphere for 2 h. The reaction medium was filtered on dicalite (methanol rinsing). The filtrate was concentrated under reduced pressure to give (4) (849 mg; 95%) in the form of a beige solid.

¹H NMR (300 MHz, DMSO) δ 7.78 (s; 2H_{2,4}); 5.54 (s broad; 2H_{9,11}); 4.59 (5; 4H_{8,10}).

¹³C NMR (75 MHz, DMSO) δ 166.7 (1C₆); 162.5 (2C_{1,5}); 139.4 (1C₃); 117.3 (2C_{2,4}); 64.0 (2C_{8,10}).

HRMS (ESI): neutral mass calculated for C₈H₉NO₄[M]: 183.0532; observed 183.0526.

Preparation of methyl 6-((2,6-bis(hydroxymethyl)pyridin-4-yl)amidohexanoate (5)

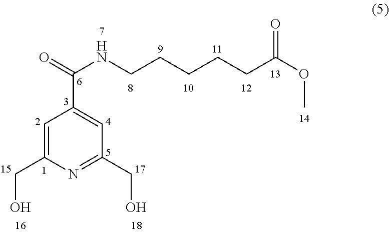 [SMILES: COC(=O)CCCCCNC(=O)c1cc(CO)nc(CO)c1]

2,6-bis(hydroxymethyl)isonicotinic acid (4) (50 mg; 0.273 mmol; 1 eq) was dissolved in anhydrous N,N-dimethylformamide (3.0 mL), the solution was cooled to 0° C., and then HATU (156 mg; 0.410 mmol; 1.5 eq) and 2,6-lutidine (147.0 μL; 1.260 mmol; 4.7 eq) were added. The activation solution was stirred at 0° C. for 15 min and then methyl 6-aminohexanoate (59 mg; 0.322 mmol; 1.2 eq) was added. The walls of the flask were rinsed with 2 mL of anhydrous N,N-dimethylformamide and the reaction medium was stirred at room temperature for 15 h. The reaction mixture was diluted in ethyl acetate, washed three times with a saturated sodium chloride solution, dried over magnesium sulfate, filtered and concentrated under reduced pressure. The product was purified by flash chromatography (dichloromethane/methanol, 90:10) to give (5) (76 mg; 91%) in the form of an off-white solid.

¹H NMR (300 MHz, DMSO) δ 8.79 (t; J=5.6 Hz; 11-₁₇); 7.71 (s; 2H_{2,4}); 5.50 (t; J=5.8 Hz; 2H16,18); 4.57 (d; J=5.8 Hz; 4H_{15,17}); 3.57 (s; 3H₁₄); 3.25 (m; 2H₈); 2.30 (t; J=7.4 Hz; 2H₁₂); 1.62-0 1.45 (m; 4H_{9,11}); 1.37-1.21 (m; 2H₁₀).

¹³C NMR (75 MHz, DMSO) δ 173.3 (1C₁₃); 165.1 (106); 161.8 (21,5); 142.9 (1C₃); 115.8 (2C_{2,4}); 64.1 (2C_{15,17}); 51.2 (1C₁₄); 38.5 under DMSO (1C₈); 33.2 (1C₁₂); 28.6 (1C₉); 25.9 (1C-H); 24.2 (1C₁₀).

HRMS (ESI): neutral mass calculated for C₁₅H₂₂N₂O₅[M]: 310.1529; observed 310.1526.

Preparation of methyl 6-(2,6-bis(bromomethyl)pyridin-4-yl)amidohexanoate (6)

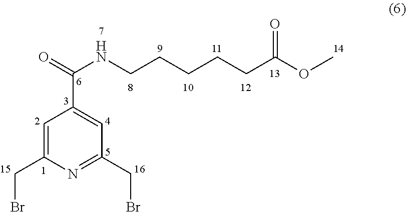 [SMILES: COC(=O)CCCCCNC(=O)c1cc(CBr)nc(CBr)c1]

Methyl 6-((2,6-bis(hydroxymethyl)pyridin-4-yl)amidohexanoate (5) (55 mg; 0.177 mmol; 1 eq) was suspended in anhydrous acetonitrile (10.5 mL) and then phosphorus tribromide (50 pL; 0.532 mmol; 3.0 eq) was added dropwise. The reaction medium was stirred at 45° C. for 2 h. The solution was cooled to 0° C., neutralized with water (10 mL) and extracted with ethyl acetate (3×15 mL). The combined organic phases were washed with a saturated sodium chloride solution, dried over magnesium sulfate and concentrated under reduced pressure. The product was purified by flash chromatography (SiO₂, cyclohexane/ethyl acetate 60:40) to give (6) (57 mg; 74%) in the form of a white solid.

¹H NMR (300 MHz; DMSO) δ 8.83 (t, J=5.6 Hz;1H₇); 7.84 (s; 2H_{2,4}); 4.74 (s; 4H_{15,16}); 3.57 (s, 3H₁₄); 3.31-3.20 (m; 2H₈); 2.31 (t; J=7.4 Hz; 2H₁₂); 1.64-1.45 (m; 4H_{9,11}); 1.39-1.22 (m, 2H₁₀).

¹³C NMR (75 MHz, DMSO) δ 173.3 (1C₁₃); 163.8 (1C₆); 157.5 (2C_{1,5}); 144.2 (1C₃); 120.8 (2C_{2,4}); 51.2 (1C₁₄); 38.9 under DMSO (1C₈); 34.1 (2C_{15,16}); 33.2 (1C₁₂); 28.5 (1C₉); 25.9 (1C₁₁); 24.2 (1C₁₀).

HRMS (ESI): neutral mass calculated for C₁₅H₂₀Br₂N₂O₃ [M]: 433.9841; observed 433.9832.

Preparation of 6-(2,6-bis(bromomethyl)pyridin-4-yl)amidohexanoic acid (⁷)

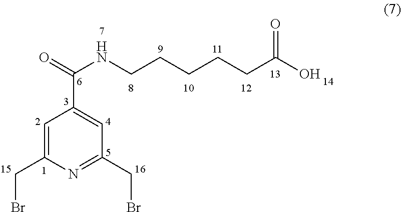 [SMILES: O=C(O)CCCCCNC(=O)c1cc(CBr)nc(CBr)c1]

Methyl 6-(2,6-bis(bromomethyl)pyridin-4-yl)amidohexanoate (6) (57 mg; 0.131 mmol; 1.0 eq) was dissolved in tetrahydrofuran (4 mL) and a solution of hydrated lithium hydroxide (8 mg; 0.327 mmol; 2.5 eq) in water (4 mL) was added slowly. The reaction medium was stirred at room temperature for 8.5 h. The tetrahydrofuran was evaporated under reduced pressure and the aqueous residue was treated with an aqueous 1N hydrochloric acid solution and extracted with ethyl acetate (3x10 mL). The combined organic phases were washed with a saturated sodium chloride solution, dried over magnesium sulfate and concentrated under reduced pressure. The product was purified by flash chromatography (dichloromethane/methanol, 90:10) to give (7) (44 mg; 80%) in the form of an off-white solid.

¹H NMR (300 MHz; DMSO) δ 12.00 (s; 1H₁₄); 8.83 (t; J=5.5 Hz; 1H₇); 7.84 (s; 2H_{2,4}); 4.74 (s; 4H_{15,17}); 3.31-3.21 (m; 2H₈); 2.21 (t; J=7.3 Hz; 2H₁₂); 1.60 -1.46 (m; 4H_{9,11}); 1.39-1.25 (m; 2H₁₀).

¹³C NMR (75 MHz; DMSO) δ 174.4 (1C₁₃); 163.8 (1C₆); 157.5 (2C_{1,5}); 144.1 (1C₃); 120.8 (2C_{2,4}); 39.0 under DMSO (1C₈); 34.1 (2C_{15,16}); 33.6 (1C₁₂); 28.6 (1C₉); 26.0 (1C₁₁); 24.2 (1C₁₀).

HRMS (ESI): m/z calculated for C₁₄H₁₉Br₂N₂O₃ [M+H]⁺: 420.9757; observed 420.9752.

Preparation of 6-(2,6-bis(bromomethyl)pyridin-4-yl)amido-N-hexanamide-valine-citrulline-p-aminobenzylcarbamate-MMAE (8)

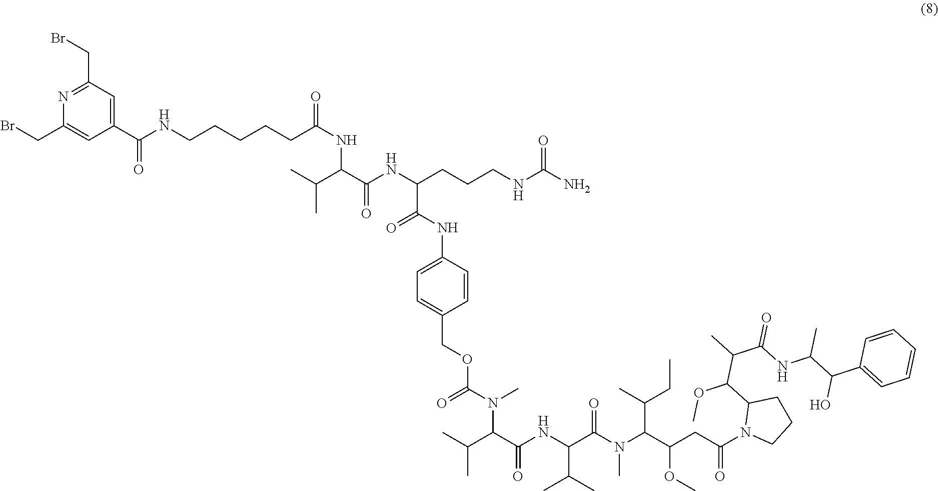 [SMILES: CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)OCc1ccc(NC(=O)C(CCCNC(N)=O)NC(=O)C(NC(=O)CCCCCNC(=O)c2cc(CBr)nc(CBr)c2)C(C)C)cc1)C(C)C]

Under an inert atmosphere, in the dark and under anhydrous conditions, 6-(2,6-bis(bromomethyl)pyridin-4-yl)amidohexanoic acid (7) (13.2 mg; 0.0313 mmol; 2.28 eq) was dissolved in anhydrous acetonitrile (1.2 mL) and then N-ethoxycarbonyl-2-ethoxy-1,2-dihydroquinoline (EEDQ) (21.2 mg; 0.0857 mmol; 6.25 eq) was added. The activation medium was stirred at 25° C. for 1 h 20. A solution of valine-citrulline-p-aminobenzylcarbamate-MMAE trifluoroacetic acid salt (17.0 mg; 0.0137 mmol; 1.0 eq), dissolved in anhydrous N,N-dimethylformamide (300 μL) in the presence of N,N-diisopropylethylamine (9.4 μL; 0.0537 mmol; 3.92 eq), was added to the activation medium. The reaction medium obtained was stirred at 25° C. for 1 h. The mixture was diluted twofold with N,N-dimethylformamide and purified by semi-preparative high-pressure liquid chromatography (t_{R}=22.1 min; on a Gilson PLC 2050 system [ARMEN V2 (pump) and ECOM TOYDAD600 (UV detector)], UV detection at 254 nm at 25° C.; Waters XBridge™ C-18 column; 5 μm (250 mm×19.00 mm); elution with 0.1% trifluoroacetic acid (by volume) in water (solvent A), and acetonitrile (solvent B); gradient 20 to 100% B over 32 min then 100% B for 6 min at 17.1 mL/min) to give (8) (18.2 mg; 87%) in the form of a white solid.

¹H NMR (300 MHz, DMSO) δ (ppm) 10.04-9.95 (m; 1H); 8.94-8.79 (m; 1 H); 8.20-8.06 (m; 2H); 7.98-7.87 (m; 1 H); 7.84 (s; 2H); 7.81 (s; 1 H); 7.70-7.61 (m; 1 H); 7.58 (d; J=8.2 Hz; 2H); 7.38-7.11 (m; 6H); 6.07-5.92 (m; 1 H); 5.47-5.37 (m; 1 H); 5.15-4.96 (m; 1 H); 4.73 (s; 4H); 4.54-4.29 (m; 2H); 4.32-4.12 (m; 1 H); 4.05-3.92 (m; 1 H); 3.30-3.08 (m; 9H); 3.06-2.93 (m; 2H); 2.91-2.77 (m; 2H); 2.24-2.05 (m; 2H); 2.21-2.11 (m; 3H); 2.02-1.88 (m; 1 H); 1.60 -1.44 (m; 5H); 1.36-1.13 (m; 4H); 1.08-0.93 (m; 6H); 0.93-0.67 (m; 28H).

HRMS (ESI): m/z calculated for C₇₂H₁₁₁Br₂N₁₂O₁₄ [M+H]+: 1525.6704; observed 1525.6700.

### Example 1B: Synthesis of a Cytotoxic Conjugate According to the Invention (Pyridine Retroamide)

General reaction scheme

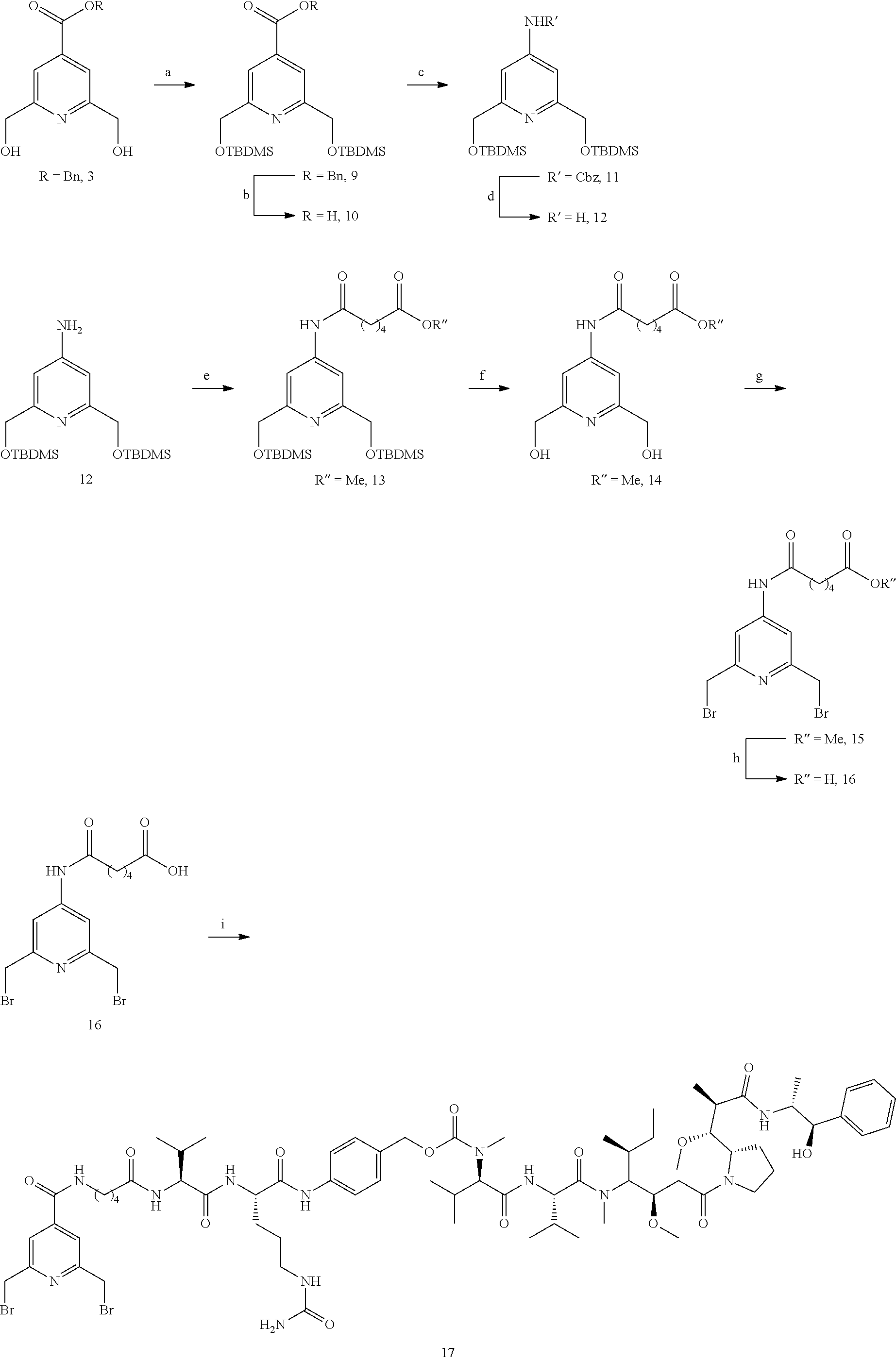

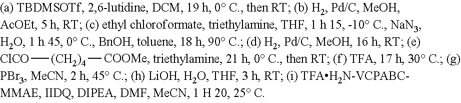

Detailed reaction scheme

Preparation of benzyl 2,6-bis(((tert-butyldimethylsilyl)oxy)methyl)isonicotinate (9)

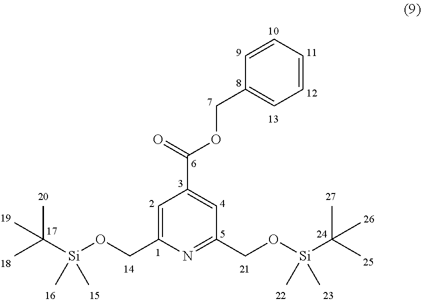 [SMILES: CC(C)(C)[Si](C)(C)OCc1cc(C(=O)OCc2ccccc2)cc(CO[Si](C)(C)C(C)(C)C)n1]

Benzyl 2,6-bis(hydroxymethyl)isonicotinate (3) (1.56 g; 5.708 mmol; 1.0 eq) was dissolved in anhydrous dichloromethane (12 mL), 2,6-lutidine (3.6 mL; 28.542 mmol; 5.0 eq) was added and the solution was cooled to 0° C. tert-Butyldimethylsilyl trifluoromethanesulfonate (5.5 mL; 23.974 mmol; 4.2 eq) was added dropwise in 10 min, and then the reaction medium was stirred under argon at room temperature (20° C.) for 19 h. The medium was cooled to 0° C. and then neutralized by addition of a saturated sodium hydrogen carbonate solution. The aqueous phase was extracted with dichloromethane (3x100 mL) and the combined organic phases were washed with a saturated sodium chloride solution, dried over magnesium sulfate, filtered and then concentrated under reduced pressure. The crude product was purified by flash chromatography (SiO₂, cyclohexane/ethyl acetate 90:10) to give (9) (2.50 g; 87%) in the form of a beige solid.

¹H NMR (300 MHz, DMSO) δ 7.83 (s; 2H_{2,4}); 7.50-7.36 (m, 5H₉₋₁₃); 5.38 (s; 2H₇); 4.79 (s; 4H_{14,21}); 0.90 (s; 18H_{18-20,25-27}); 0.09 (s; 12H_{16,15,22,23}).

Preparation of 2,6-bis(((tert-butyldimethylsilyl)oxy)methyl)isonicotinic acid (10)

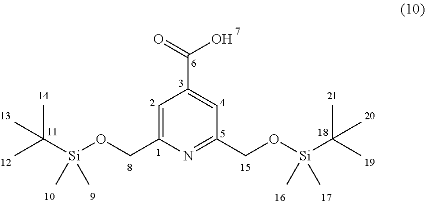 [SMILES: CC(C)(C)[Si](C)(C)OCc1cc(C(=O)O)cc(CO[Si](C)(C)C(C)(C)C)n1]

Benzyl 2,6-bis(((tert-butyldimethylsilyl)oxy)methyl)isonicotinate (9) (2.50 g; 4.982 mmol; 1.0 eq) was dissolved in 60 mL of a methanol/ethyl acetate mixture (5:1) and the solution was degassed with argon for 15 min. 10% by weight palladium-on-charcoal (250 mg, 10% m/m) was added and the reaction medium was stirred at room temperature (20° C.) under a hydrogen atmosphere for 5 h. The reaction medium was filtered on dicalite (methanol rinsing). The filtrate was concentrated under reduced pressure to give (10) (1.93 g; 94%) in the form of a white solid.

¹H NMR (300 MHz, DMSO) δ 7.78 (s; 2H_{2,4}); 4.78 (s; 4H-_{18,15}), 0.92 (s; 18H_{12-14,19-21}); 0.10 (s; 12H9,10,16,17).

Preparation of benzyl (2,6-bis(((tert-butyldimethylsilyi)oxy)methyl)pyridin-4-yl)carbamate (11)

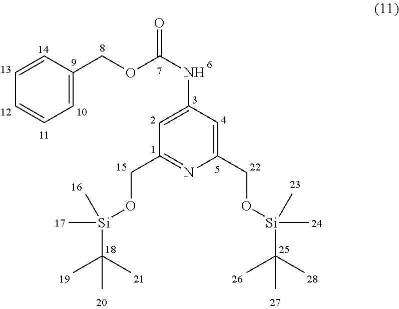 [SMILES: CC(C)(C)[Si](C)(C)OCc1cc(NC(=O)OCc2ccccc2)cc(CO[Si](C)(C)C(C)(C)C)n1]

2,6-bis(((tert-Butyldimethylsilyl)oxy)methyl)isonicotinic acid (10) (519.4 mg; 1.262 mmol; 1.0 eq) was dissolved in anhydrous tetrahydrofuran (4 mL). The solution was cooled to −10° C. Triethylamine (227.0 μL; 1.637 mmol; 1.3 eq) and then ethyl chloroformate (180.7 μL; 1.890 mmol; 1.5 eq) were added with stirring. The reaction medium was stirred under argon at —10° C. for 1 h 15. Then, a solution of sodium azide (139.8 mg; 2.142 mmol; 1.7 eq) in water (300 μL) was added. The reaction medium was stirred at 0° C. for 1 h 45. The triethylamine salts were filtered off, then the filtrate was concentrated under reduced pressure (maximum bath temperature 40° C., maximum vacuum 100 mbar). The residue was then taken up in 10 mL of water and the product was extracted with ethyl acetate (3×10 mL). The combined organic phases were washed with a saturated sodium chloride solution, dried over magnesium sulfate and concentrated under reduced pressure. To give the acyl azide in the form of a yellow oil. It was dissolved in toluene (14 mL), and then benzyl alcohol (522 μL; 5.044 mmol; 4.0 eq) was added. The reaction medium was stirred at 90° C. for 18 h. The toluene was evaporated under reduced pressure. The product was purified by flash chromatography (SiO₂, cyclohexane/ethyl acetate 90:10) to give (11) (535.6 mg; 82%) in the form of a yellow oil.

¹H NMR (300 MHz, CDCI3) δ 7.50-7.32 (m; 7H_{2,4,16-14}); 6.85 (s; 1H₆); 5.23 (s; 2H₈); 4.75 (s; 4H_{15,22}); 0.96 (s; 18H_{19-21,26-28}); 0.12 (s; 12H_{16,17,23,24}).

Preparation of 2,6-bis(((tert-butyldimethylsilyl)oxy)methyl)pyridin-4-amine (12)

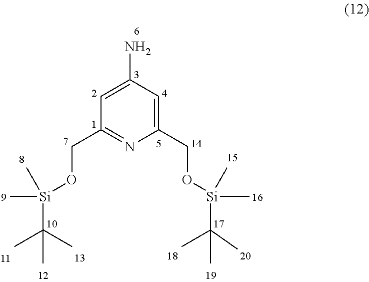

Benzyl (2,6-bis(((tert-butyldimethylsilyl)oxy)methyl)pyridin-4-yl)carbamate (11) (528.6 mg; 1.020 mmol; 1.0 eq) was dissolved in methanol (30 mL). The solution was degassed with argon for 15 min. Then, 10% by weight palladium-on-charcoal (57.4 mg, 10% m/m) was added. The reaction medium was stirred under a hydrogen atmosphere at room temperature (20° C.) for 16 h. The palladium on charcoal was filtered on dicalite (methanol rinsing) and then the filtrate was concentrated under reduced pressure. The product was obtained in salified form (nitrogen of the pyridine). This was taken up in water (160 mL), and then a 10% solution of sodium hydroxide in water was added at 0° C. until a pH of 9 was obtained. The product was then extracted with dichloromethane (5×50 mL). The combined organic phases were washed with a saturated sodium chloride solution, dried over magnesium sulfate and concentrated under reduced pressure to give (12) (312.2 mg; 80%) in the form of a yellow oil.

¹H NMR (300 MHz; DMSO) δ 6.44 (s; 2H_{2,4}); 6.04 (s; 2H₆); 4.47 (s; 4H_{7,14});

0.91 (s; 18H_{11-13,18-20}); 0.07 (s; 12H_{8,9,15;16}).

Preparation of methyl 6-((2,6-bis(((tert-butyldimethylsilyl)oxy)methyl)pyridin-4-yl)amino)-6-oxohexanoate (13)

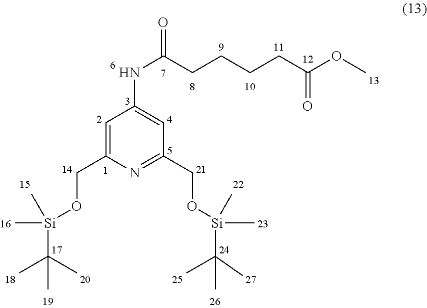

2,6-bis(((tert-Butyldimethylsilyl)oxy)methyl)pyridin-4-amine (12) (146.7 mg; 0.383 mmol; 1.0 eq) was dissolved in anhydrous dichloromethane (2.8 mL). The solution was cooled to 0° C. and triethylamine (106.7 pL; 0.765 mmol; 2.0 eq) and then methyl adipoyl chloride (59.7 μL; 0.383 mmol; 1.0 eq) were added dropwise. The reaction medium was stirred at room temperature (20° C.) for 15 h 30. Anhydrous dichloromethane (1 mL) was then added to the reaction medium, as was methyl adipoyl chloride (26.8 μL; 0.172 mmol; 0.5 eq). The reaction medium was stirred at room temperature (20° C.) for 1 h 30. Then, anhydrous dichloromethane (1 mL) was added to the reaction medium, as were methyl adipoyl chloride (59.7 μL; 0.383 mmol; 1.0 eq) and triethylamine (26.7 μL; 0.191 mmol; 0.5 eq). The reaction medium was stirred at room temperature (20° C.) for 4 h. The reaction was halted by addition of a saturated sodium hydrogen carbonate solution (3 mL) at 0° C. and extracted with dichloromethane (3×10 mL). The combined organic phases were dried over magnesium sulfate and concentrated under reduced pressure. The product was purified by flash chromatography (SiO2, cyclohexane/ethyl acetate 50:50) to give (13) (144.0 mg; 72%) in the form of a colorless oil.

¹H NMR (300 MHz, DMSO) δ 10.25 (s; 1H₆); 7.58 (s; 2H_{2,4}); 4.63 (s; 4H_{14,21}); 3.58 (s; 3H13); 2.41-2.28 (m; 4118,11); 1.62-1.51 (m; 4H_{9,10}); 0.92 (s, 18H_{18-20,25-27);} 0.09 (s; 12H_{15,16,22,23}).

Preparation of methyl 6-((2,6-bis(hydroxymethyl)pyridin-4-yl)amino)-6-oxohexanoate (14)

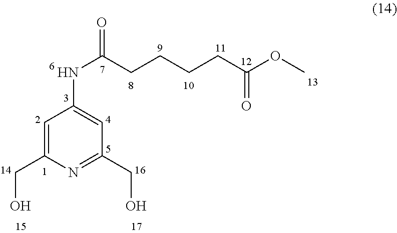 [SMILES: COC(=O)CCCCC(=O)Nc1cc(CO)nc(CO)c1]

Methyl 6-((2,6-bis(((tert-butyldimethylsilyl)oxy)methyl)pyridin-4-yl)amino)-6-oxohexanoate (13) (136.9 mg; 0.261 mmol; 1.0 eq) was dissolved in trifluoroacetic acid (3 mL). The reaction medium was stirred at 30° C. for 17 h. The trifluoroacetic acid was evaporated under reduced pressure. The residue was taken up in ethyl acetate (10 mL) and a saturated sodium hydrogen carbonate solution (10 mL) was added. The emulsion was stirred vigorously. The product was extracted with ethyl acetate (5×10 mL). The combined organic phases were dried over magnesium sulfate and concentrated under reduced pressure. The product was purified by flash chromatography (SiO₂, dichloromethane/methanol 80:20) to give (14) (72.2 mg; 93%) in the form of a colorless oil.

¹H NMR (300 MHz, DMSO) δ 10.24 (s; 1H₆); 7.57 (s; 2H_{2,4}); 5.37 (t; J=5.8

Hz; 2H_{15,17}); 4.45 (d; J=5.8 Hz; 4H_{14,16}); 3.58 (s; 3H₁₃); 2.40-2.29 (m; 4118,11); 1.72-1.45 (m; 4H_{9,10}).

Preparation of methyl 6-((2,6-bis(bromomethyl)pyridin-4-yl)amino)-6-oxohexanoate (15)

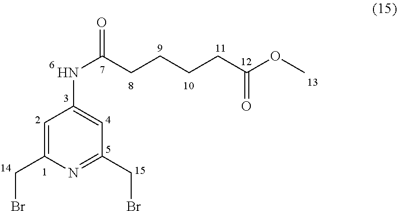 [SMILES: COC(=O)CCCCC(=O)Nc1cc(CBr)nc(CBr)c1]

Methyl 6-((2,6-bis(hydroxymethyl)pyridin-4-yl)amino)-6-oxohexanoate (14) (93.5 mg; 0.316 mmol; 1.0 eq) was dissolved in anhydrous acetonitrile (6 mL) and then phosphorus tribromide (90 μL; 0.958 mmol; 3.0 eq) was added slowly. The reaction medium was stirred at 45° C. for 2 h. The solution was cooled to 0° C., neutralized with water (5 mL) and extracted with ethyl acetate (3×10 mL). The combined organic phases were washed with a saturated sodium chloride solution, dried over magnesium sulfate and concentrated under reduced pressure. The product was obtained in salified form (nitrogen of the pyridine). This was taken up in water, and then a 10% solution of sodium hydroxide in water was added at 0° C. until a pH of 9 was obtained. The product was then extracted with dichloromethane (3×20 mL). The combined organic phases were washed with a saturated sodium chloride solution, dried over magnesium sulfate and concentrated under reduced pressure. The product was purified by flash chromatography (SiO₂,dichloromethane/methanol 90:10) to give (15) (80.8 mg; 62%) in the form of a slightly pink solid.

¹H NMR (300 MHz, CDCI₃) δ 7.87 (s; 1H₆); 7.64 (s; 2H_{2,4}); 4.49 (s; 4H_{14,15}); 3.71 (s; 3H₁₃); 2.53-2.32 (m; 4H_{8,11}); 1.84-1.66 (m; 4H_{9,10}).

Preparation of 6-((2,6-bis(bromomethyl)pyridin-4-yl)amino)-6-oxohexanoic acid (16)

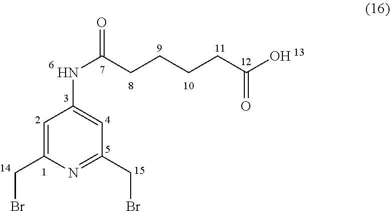 [SMILES: O=C(O)CCCCC(=O)Nc1cc(CBr)nc(CBr)c1]

Methyl 6-((2,6-bis(bromomethyl)pyridin-4-yl)amino)-6-oxohexanoate (15) (35.2 mg; 0.083 mmol; 1.0 eq) was dissolved in tetrahydrofuran (2.2 mL) and a solution of hydrated lithium hydroxide (8.7 mg; 0.208 mmol; 2.5 eq) in water (2.1 mL) was added. The reaction medium was stirred at room temperature (20° C.) for 3 h. The reaction medium was acidified at 0° C. with an aqueous 0.1 N hydrochloric acid solution and then extracted with ethyl acetate (3×20 mL). The combined organic phases were washed with a saturated sodium chloride solution, dried over magnesium sulfate and concentrated under reduced pressure lo to give (16) (33.4 mg; 99%) in the form of a crystalline white solid.

¹H NMR (300 MHz, DMSO) δ 12.06 (s; 1H₁₃); 10.46 (s; 1H₆); 7.66 (s; 2H_{2,4}); 4.63 (s; 4H_{14,15}); 2.39-2.33 (m; 2H₈); 2.26-2.20 (m; 2H₁₁); 1.67-1.45 (m; 4H_{9,10}).

Preparation of 6-((2,6-bis(bromomethyl)pyridin-4-yl)amino)-6-oxohexanamide-valine-citrulline-p-aminobenzylcarbamate-MMAE (17)

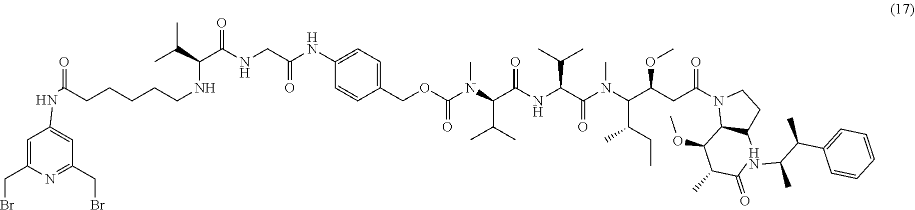

Under an inert atmosphere, in the dark and under anhydrous conditions, 6-((2,6-bis(bromomethyl)pyridin-4-yl)amino)-6-oxohexanoic acid (16) (3.1 mg; 7.60 μmol; 1.8 eq) was dissolved in anhydrous acetonitrile (110 μL) and then isobutyl 1,2-dihydro-2-isobutoxy-1-quinolinecarboxylate (IIDQ) (2.28 μL; 7.68 μmol; 1.8 eq) was added. The activation medium was stirred at 25° C. for 30 min.

A solution of valine-citrulline-p-aminobenzylcarbamate-MMAE trifluoroacetic acid salt (5.3 mg; 4.28 μmol; 1.0 eq), dissolved in anhydrous N,N-dimethylformamide (200 μL) in the presence of N,N-diisopropylethylamine (2.98 μL; 17.11 μmol; 4.0 eq) and previously stirred at room temperature (20° C.) for 30 min, was added to the activation medium. The reaction medium obtained was stirred at 25° C. for 1 h 20. The mixture was diluted twofold with acetonitrile and purified by semi-preparative high-pressure liquid chromatography (t_{R}=23.5 min; on a Gilson PLC 2050 system [ARMEN V2 (pump) and ECOM TOYDAD600 (UV detector)], UV detection at 254 nm at 25° C.; Waters XBridge™ C-18 column; 5 μm (250 mm×19.00 mm); elution with 0.1% trifluoroacetic acid (by volume) in water (solvent A), and acetonitrile (solvent B); gradient 30 to 60% B over 26 min then 60% to 100% B over 2 min and 100% B for 3 min at 17.1 mL/min) to give (17) (2.7 mg; 42%) in the form of a white lyophilizate.

¹H NMR (300 MHz, CDCI₃): δ 7.97 (5; 1 H); 7.58-7.41 (m; 1 H); 7.40-7.29 (m; 5H); 5.42-5.14 (m; 1 H); 5.00-4.85 (m; 2H); 4.76-4.64 (m; 1 H); 4.63-4.45 (m; 5H); 4.23-4.01 (m; 1 H); 4.02-3.72 (m; 2H); 3.60-3.43 (m; 3H); 3.43-3.35 (m; 3H); 3.34-3.25 (m; 4H); 3.23-3.08 (m; 4H); 3.07-2.97 (m; 3H); 2.95-2.81 (m; 3H); 2.62-2.29 (m; 6H); 1.84-1.40 (m; 21 H); 1.35-1.11 (m; 6H); 1.10-0.92 (m; 17H); 0.93-0.69 (m; 16H); 0.71-0.53 (m; 3H).

HRMS (ESI): m/z calculated for C₇₁ H₁₀₉Br₂N₁₂O₁₄ [M+H]+: 1511.6547; observed 1511.6557.

### Example 2A: Synthesis of an Antibody-Drug Conjugate According to the Invention (Pyridine)

Code of the synthesized product: McSAF-pyridine (corresponding to formula

(Ila), also called hereinafter “antibody-drug conjugate according to the invention” or generally “ADC”).

Antibody used: trastuzumab.

Preparation of solutions

Bioconjugation buffer: 1X saline buffer, for example phosphate, borate, acetate, glycine, tris(hydroxymethyl)aminomethane, 4-(2-hydroxyethyl)-1-piperazineethanesulfonic acid in a pH range of between 6 and 9, with a final NaCI concentration of between 50 and 300 mM and a final EDTA concentration of io between 0.1 and 10 mM. For example, 1X phosphate buffer at a pH of 8.3, with a final NaCI concentration of 180 mM and a final EDTA concentration of 1 mM.

Trastuzumab at a concentration of between 1 and 10 mg/mL in the bioconjugation buffer, for example 5 mg/mL.

Reducing agent: Solution of a reducing agent chosen from dithiothreitol, (3-mercaptoethanol, tris(2-carboxyethyl)phosphine hydrochloride, tris(hydroxypropyl)phosphine at a concentration of between 0.1 and 10 mM in the bioconjugation buffer. For example, a 1 mM solution of tris(2-carboxyethyl)phosphine hydrochloride in the bioconjugation buffer.

Linker solution: cytotoxic conjugate (8) at a concentration of between 0.1 and 10 mM in a mixture of organic solvents chosen from dimethyl sulfoxide, N,N-dimethylformamide, methanol, tetrahydrofuran, acetonitrile, N,N-dimethylacetamide, dioxane. For example, a 1 mM solution in a mixture of organic solvents composed of 20% N,N-dimethylformamide and 80% methanol.

Bioconjugation reaction

Under an inert atmosphere, the reducing agent (4 to 100 eq, for example 8 eq) was added to the trastuzumab in the bioconjugation buffer (2.5 mg; 1 eq) and the reaction medium was incubated between 15 and 40° C., for example 37° C., for 0.25 to 3 h, for example 2 h, and then the linker solution (4 to 100 eq, for example 12 eq) was added under an inert atmosphere and the reaction medium was agitated between 15 and 40° C., for example 37° C., for 0.5 to 15 h, for example 2.5 h. This reaction was duplicated, in parallel, as many times as necessary to obtain the desired final amount of ADC, i.e. five times.

Purification of the ADC

The reaction mixture was purified on a PD-10 (GE Healthcare) using Gibco PBS pH 7.4 buffer as many times as necessary to remove residual chemical reagents, i.e. purified two times.

Results

The steps described above made it possible to obtain 7.43 mg of McSAF-pyridine (57%).

### Example 2B: Synthesis of an Antibody-Drug conjugate According to the Invention (Pyridine Retroamide)

Code of the synthesized product: McSAF-pyridine retroamide (corresponding to formula (Ila), also called hereinafter “antibody-drug conjugate according to the invention” or generally “ADC”).

Antibody used: trastuzumab.

Preparation of solutions

Bioconjugation buffer: 1X borate buffer at a pH of 8.3, with a final NaCI concentration of 25 mM and a final EDTA concentration of 1 mM.

Trastuzumab at a concentration of 5 mg/mL in the bioconjugation buffer.

Reducing agent: Solution of tris(2-carboxyethyl)phosphine hydrochloride at a concentration of 1 mM in the bioconjugation buffer.

Linker solution: cytotoxic conjugate (17) at a concentration of 2 mM in a mixture of organic solvents composed of 70% N,N-dimethylformamide and 30% methanol.

Bioconjugation reaction

The solution of trastuzumab in the bioconjugation buffer (0.25 mg; 1 eq) was placed under argon. The reducing agent (8 eq) was then added and the reaction medium was incubated at 37° C. for 2 h. Then, the linker solution (17 eq) was added under argon and the reaction medium was agitated at 37° C. for 2 h 30.

### Example 3A: analyses of the antibody-drug conjugate (McSAF-pyridine)

HIC (hydrophobic interaction chromatography) analysis

Materials and method

The McSAF-pyridine ADC was diluted to 1 mg/mL with PBS pH 7.4 before being filtered through 0.22 pm. 50 pg of products were injected onto an MAbPac

HIC-Butyl, 5 pm, 4.6 x 100 mm, column (ThermoScientific), connected to a Waters Alliance HPLC system (e2695) equipped with a PDA (e2998) set for detection at 280 nm. The McSAF-pyridine ADC was eluted at a flow rate of 1 mL/min with a gradient from 100% buffer A (1.5 M ammonium sulfate, 50 mM monobasic sodium phosphate, 5% isopropanol (v/v), pH 7.0) to 20% buffer B (50 mM monobasic sodium phosphate, 20% isopropanol (v/v), pH 7.0) in 2 minutes then to 85% buffer B in 30 minutes and then this gradient was maintained for 1 min. The temperature was maintained at 25° C. throughout the separation.

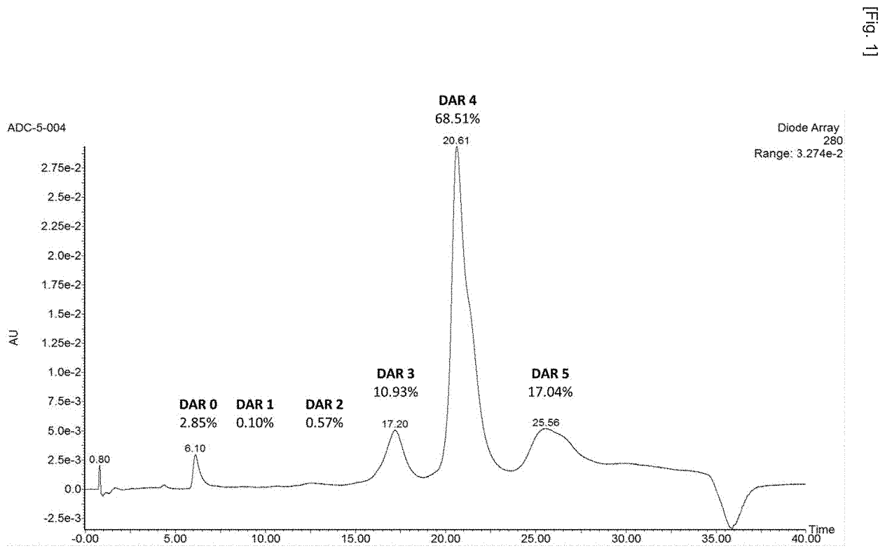

The results are shown in FIG. 1 and in table 1 below.

TABLE 1

| McSAF-pyridine | DAR 0 | DAR 1 | DAR 2 | DAR 3 | DAR 4 | DAR 5 |
|---|---|---|---|---|---|---|
| Retention time (min) | 6.1 | 9.73 | 12.59 | 17.2 | 20.61 | 25.56 |
| Area (%) | 2.85 | 0.1 | 0.57 | 10.93 | 68.51 | 17.04 |
| Average DAR | 3.93 | | | | | |

SEC (size exclusion chromatography) analysis

Materials and method

The McSAF-pyridine ADC was diluted to 1 mg/mL with PBS pH 7.4 before being filtered through 0.22 μm. 50 _{i—}ig of products were injected onto an AdvanceBio SEC 2.7 μm, 7.8×300 mm, column (Agilent Technologies), connected to a Waters Alliance HPLC system (e2695) equipped with a PDA (2998) set for detection at 280 nm, with a MALLS Wyatt (miniDawn™) detector and with a Wyatt (Optilab® T-rEX) refractive index detector. The McSAF-pyridine ADC was eluted at a flow rate of 1 mL/min with an isocratic buffer C (1 mM monobasic sodium phosphate, 155 mM sodium chloride, 3 mM dibasic sodium phosphate, 3 mM sodium azide, pH 7.0) in 24 minutes. The temperature was maintained at 25° C. throughout the separation.

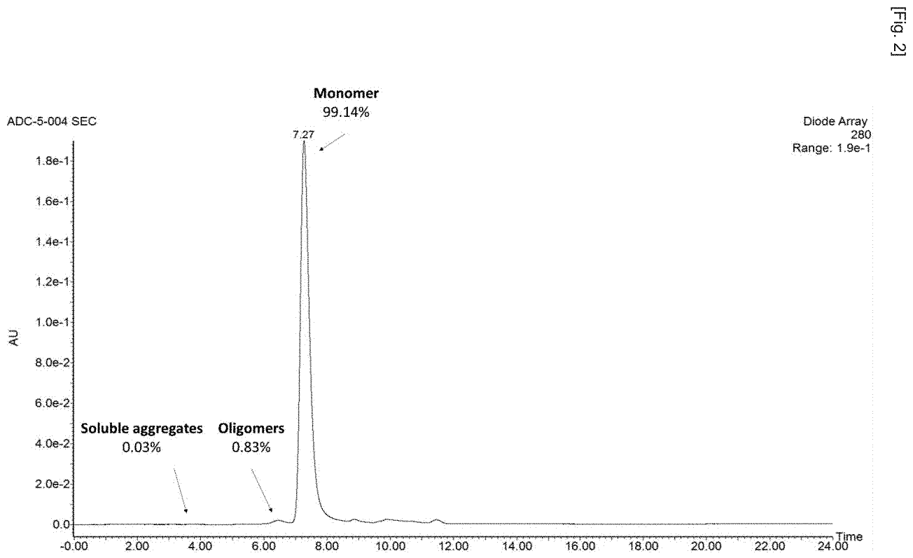

The results are shown in FIG. 2 and in table 2 below.

TABLE 2

| McSAF-pyridine | Soluble aggregates | Oligomers | Monomers |
|---|---|---|---|
| Retention time (min) | 3.73 | 6.46 | 7.27 |
| Area (%) | 0.03 | 0.83 | 99.14 |

Reproducibility

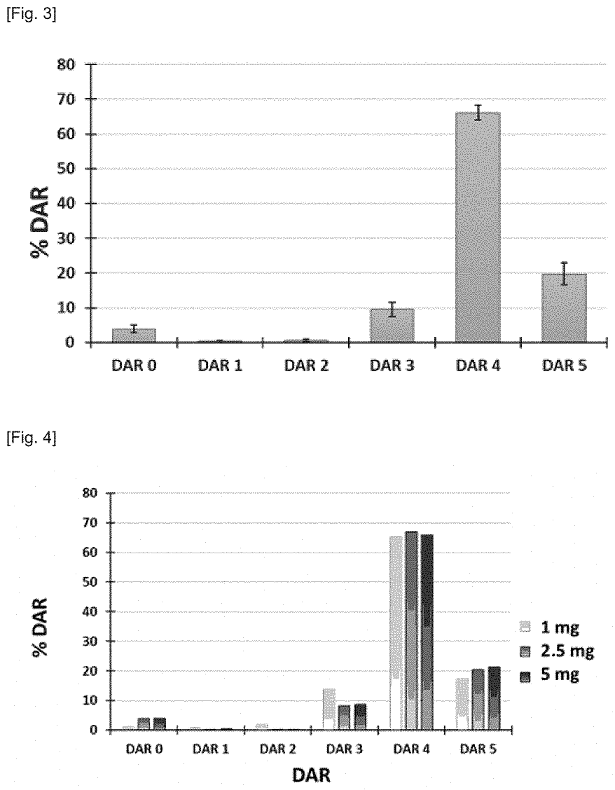

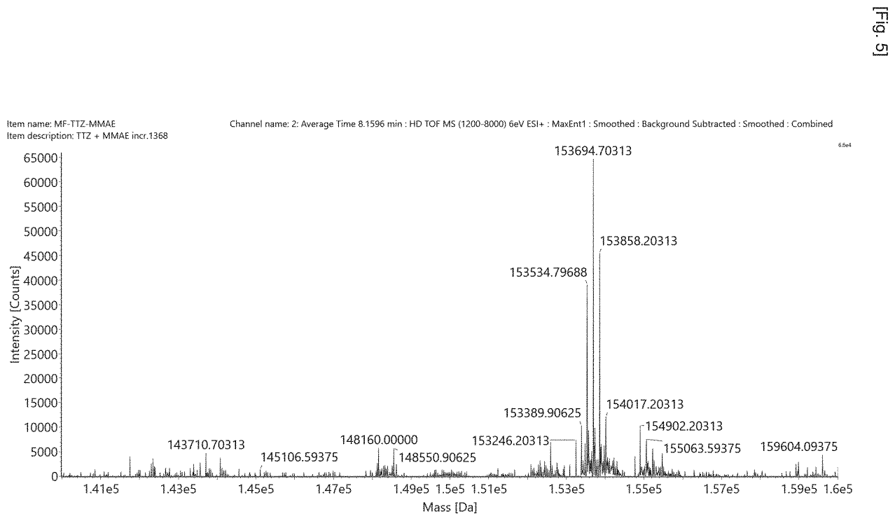

The reproducibility results are presented in FIG. 3 (reproducibility over 14 independent bioconjugations (scale 1 mg of trastuzumab employed), analysis by HIC) and in FIG. 4 (reproducibility on different scales, HIC analysis).

Conclusion: the bioconjugation is perfectly reproducible on the same scale and on different scales.

Native MS (native mass spectrometry) analysis

Materials and method

Mass spectrometric analysis was performed on a Vion IMS Qtof mass spectrometer coupled to an Acquity UPLC H-Class system from Waters (Wilmslow, UK). Prior to MS analysis, the samples (20 μg) were desalted on a BEH SEC 2.1×150 mm 300 A desalting column with an isocratic gradient (50 mM ammonium acetate, pH 6.5) at 40 4/min. A bypass valve was programmed to allow solvent to enter the spectrometer only between 6.5 and 9.5 min. MS data were acquired in positive mode with an ESI source over an m/z range of 500 to 8000 at 1 Hz and were analyzed using the UNIFI 1.9 software and the MaxEnt algorithm for deconvolution.

The results are shown in FIG. 5 and in table 3 below.

TABLE 3

| McSAF- | | | | | | |
|---|---|---|---|---|---|---|
| pyridine | DAR 0 | DAR 1 | DAR 2 | DAR 3 | DAR 4 | DAR 5 |
| MW (Da)¹ | n.o.² | n.o.² | n.o.² | 152165 | 153535 | 154903 |
| Area (%) | 0 | 0 | 0 | 8.20 | 79.57 | 12.23 |
| Average | 4.04 | | | | | |
| DAR | | | | | | |

¹molecular mass of the G0F/G0F glycoform
²not observed

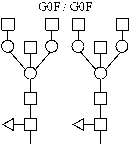

Denaturing HRMS (denaturing high resolution mass spectrometry) analysis

The mass spectrometric analysis of the McSAF-pyridine ADC was performed on a Bruker maXis mass spectrometer coupled to a Dionex Ultimate 3000 RSLC system. Prior to MS analysis, the samples (5 μg) were desalted on a MassPREP desalting column (2.1x10 mm, Waters) heated to 80° C. using an aqueous 0.1% formic acid solution as solvent A and a 0.1% solution of formic acid in acetonitrile as solvent B at 500 μL/min. After 1 min, a linear gradient of 5 to 90% B in 1.5 min was applied. MS data were acquired in positive mode with an ESI source over an m/z range of 900 to 5000 at 1 Hz and were analyzed using the DataAnalysis 4.4 software (Bruker) and the MaxEnt algorithm for deconvolution. The DAR by species was determined using the intensity of the observed peaks.

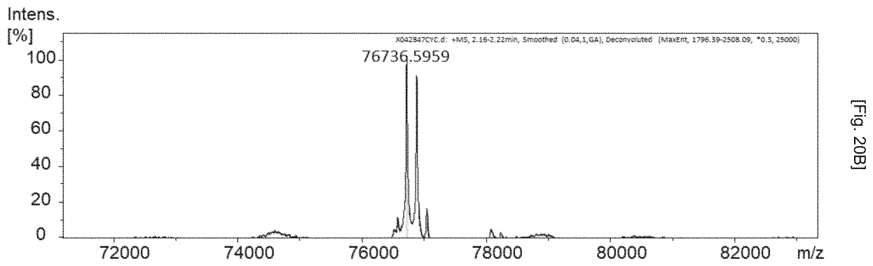

The results are shown in FIG. 18 and in table 4 below.

TABLE 4

| | LHHL | | LHH | | HH | |
|---|---|---|---|---|---|---|
| | Intensity (%) | MM (Da)¹ | Intensity (%) | MM (Da)¹ | Intensity (%) | MM (Da)¹ |
| DAR 0 | n.o.² | | n.o.² | | n.o.² | |
| DAR 1 | n.o.² | | n.o.² | | n.o.² | |
| DAR 2 | n.o.² | | n.o.² | | n.o.² | |
| DAR 3 | n.o.² | | n.o.² | | 100 | 105295 |
| DAR 4 | 86 | 153533 | 100 | 130089 | n.o.² | |
| DAR 5 | 14 | 154903 | n.o.² | | n.o.² | |
| Average DAR | 4.14 | | 4.00 | | 3.00 | |
| | | | LH | | L | |
| | | | Intensity (%) | MM (Da)¹ | Intensity (%) | MM (Da)¹ |

| | | | |
|---|---|---|---|
| DAR 0 | n.o.² | 100 | 23439 |

| DAR 1 | n.o.² | | n.o.² |
|---|---|---|---|
| DAR 2 | 100 | 76765 | n.o.² |

| | | |
|---|---|---|
| DAR 3 | n.o.² | n.o.² |
| DAR 4 | n.o.² | n.o.² |
| DAR 5 | n.o.² | n.o.² |
| Average DAR | 2.00 | 0.00 |

Species H was not observed.
¹molecular mass of the G0F/G0F glycoform
²not observed

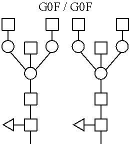

### Example 3B: analyses of the antibody-drug conjugate (McSAF-pyridine retroamide)

HIC (hydrophobic interaction chromatography) analysis

Materials and method

The McSAF-pyridine retroamide ADC was analyzed by carrying out the protocol described in example 3A.

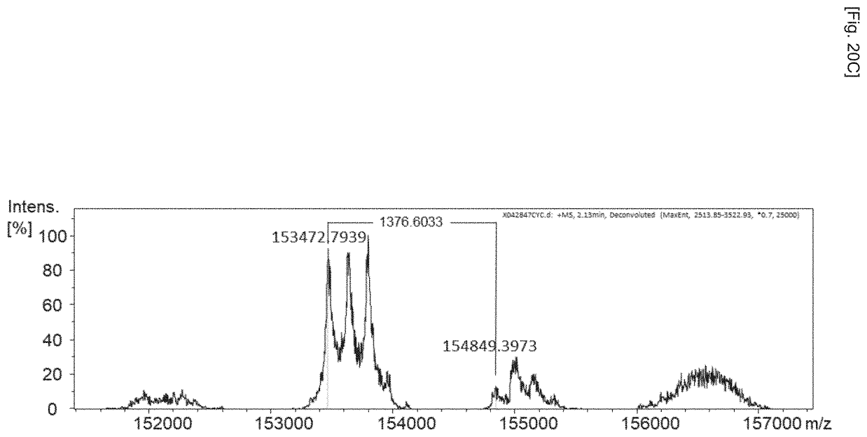

The results are shown in FIG. 19 and in table 5 below.

TABLE 5

| | McSAF-pyridine retroamide | | | | | | |
|---|---|---|---|---|---|---|---|
| | DAR 0 | DAR 1 | DAR 2 | DAR 3 | DAR 4 | DAR 5 | DAR 6 |
| Retention time (min) | 6.39 | n.o.¹ | 12.37 | 17.22 | 20.73 | 25.46 | 30.42 |
| Area (%) | 0.98 | n.o.¹ | 1.36 | 7.05 | 67.74 | 21.97 | 0.89 |
| Average DAR | 4.10 | | | | | | |

¹not observed

Denaturing HRMS (denaturing high resolution mass spectrometry) analysis

The McSAF-pyridine retroamide ADC was analyzed by carrying out the protocol described in example 3A.

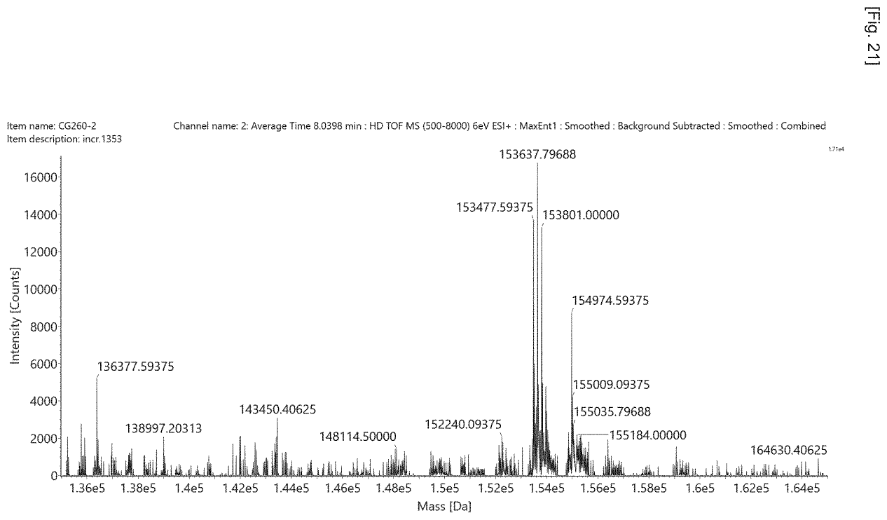

The results are shown in FIG. 20 and in table 6 below.

TABLE 6

| LHHL | | LH | | L | |
|---|---|---|---|---|---|
| Intensity (%) | MM (Da)¹ | Intensity (%) | MM (Da)¹ | Intensity (%) | MM (Da)¹ |

| | | | | |
|---|---|---|---|---|
| DAR 0 | n.o.² | n.o.² | 100 | 23439 |

| DAR 1 | n.o.² | n.o.² | | n.o.² |
|---|---|---|---|---|
| DAR 2 | n.o.² | 100 | 76737 | n.o.² |

| DAR 3 | n.o.² | | n.o.² | n.o.² |
|---|---|---|---|---|
| DAR 4 | 87 | 153473 | n.o.² | n.o.² |
| DAR 5 | 13 | 154849 | n.o.² | n.o.² |

| | | | |
|---|---|---|---|
| Average DAR | 4.13 | 2.00 | 0.00 |

The LHH, HH and H species were not observed.
¹molecular mass of the G0F/G0F glycoform
²not observed

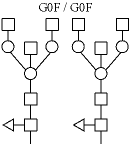

Native MS (native mass spectrometry) analysis

Materials and method

Mass spectrometric analysis was performed as described in example 3A.

The results are shown in FIG. 21 and in table 7 below.

TABLE 7

| McSAF- | | | | | | |
|---|---|---|---|---|---|---|
| pyridine | | | | | | |
| retroamide | DAR 0 | DAR 1 | DAR 2 | DAR 3 | DAR 4 | DAR 5 |
| MW (Da) | n.o.³ | n.o.³ | n.o.³ | 152240¹ | 153478² | 154841² |
| Area (%) | 0 | 0 | 0 | 4.8 | 74.5 | 20.7 |

| | |
|---|---|
| Average | 4.16 |
| DAR | |

¹molecular mass of the G0F/G1F glycoform 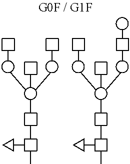
²molecular mass of the G0F/G0F glycoform 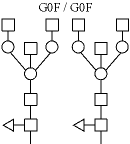
³not observed

Reproducibility

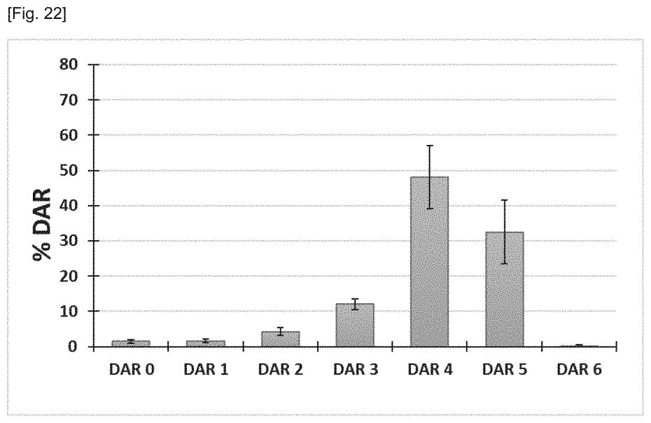

The reproducibility results are presented in FIG. 22 (reproducibility over 5 independent bioconjugations (scale 250 μg of trastuzumab employed), analysis by HIC).

### Example 4: In vitro evaluation of the antibody-drug conjugate (McSAF-pyridine)

Binding to the HER-2 antigen: recognition of the HER2 antigen on a positive line (BT-474) and a negative line (MCF-7), comparison with a reference trastuzumab-cytotoxic drug conjugate described in the prior art, called trastuzumab emtansine (hereinafter “T-DM1”).

Materials and method

The cells were obtained from the ATCC (BT-474 and MCF-7). An aliquot of frozen cells was thawed rapidly in a water bath at 37° C. and washed twice with culture medium (F12/DMEM supplemented with 8% FCS, 100 μg/mL of sodium penicillin G, 100 μg/mL of streptomycin sulfate) and placed in a 150 cm² cell culture flask at a density of at least 10 000 cells/cm². The cells were kept at 37° C. in a humid atmosphere with 5% CO₂ for at least a week.

The cells were then collected and 100 000 to 500 000 cells in 80 μL were incubated with 20 μL of ADCs (McSAF-pyridine or T-DM1) for 0.5-1 h at 4° C. at concentrations of ADCs ranging from 0.1 to 20 μg/mL (8 concentrations—0.1; 0.5; 1; 2.5; 5; 10; 15; 20 μg/mL) or with an antibody targeting HER2 (trastuzumab, positive control) at the same concentrations. The cells were washed three times with labeling buffer (1X PBS-2 mM EDTA-0.5% BSA) at 0° C. and incubated with a secondary antibody (100 μ1_{—} to 1/100th, F(ab')₂-goat anti-human IgG Fc-PE, Life Technologies, # H10104) for 0.5 — 1 h at 4° C. After incubation with the secondary antibodies, the cells were washed twice with labeling buffer at 0° C.

After washing, the cells were centrifuged and resuspended in 100-500 μL of staining buffer at 0° C. before analysis by flow cytometry. The relative mean fluorescence emission (MFI) of the probes used was determined for each sample on a flow cytometer and analyzed by software such as FCS Express 5 Flow Cytometry (De Novo Software).

Results

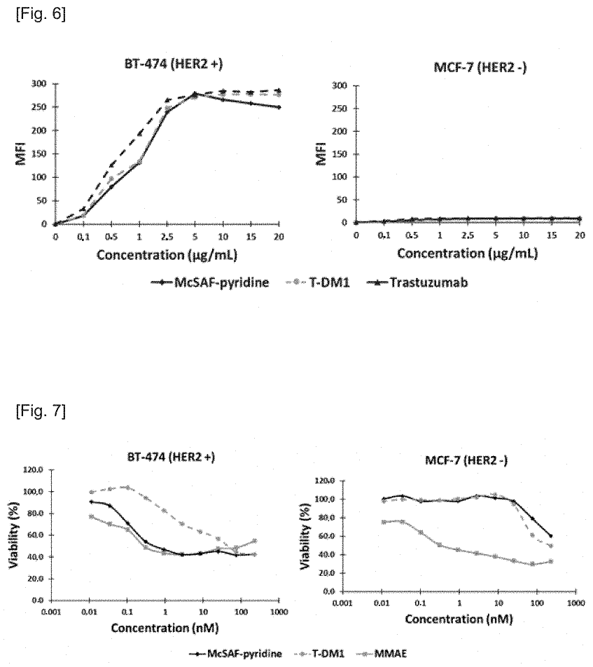

The results, presented in FIG. 6, show that the recognition of HER2 by McSAF-pyridine is similar to the recognition of HER2 by the native antibody (trastuzumab) and T-DM1, and is dependent on the presence of HER2.

Cytotoxicity (MTS assay): cytotoxic effect of McSAF-pyridine on a positive line and a negative line compared to T-DM1.

Materials and method

The cells were obtained from the ATCC (BT-474 and MCF-7). An aliquot of frozen cells was thawed rapidly in a water bath at 37° C. and washed twice with io culture medium (F12/DMEM supplemented with 8% FCS, 100 μg/mL of L-glutamine, 100 μg/mL of sodium penicillin G, 100 μg/mL of streptomycin sulfate) and placed in a 150 cm² cell culture flask at a density of at least 10 000 cells/cm². The cells were kept at 37° C. in a humid atmosphere with 5% CO₂ for at least a week.

The cells were then collected and deposited in 96-well plates at densities of between 1.25 and 2.5×10³ cells per well for the cytotoxicity assays. The cells were incubated for 48 hours at 37° C. before the addition of the ADCs tested and of the vehicle (PBS). The DMSO percentages never exceeded 0.5%. The ADCs tested were added at the following final concentrations: 225; 75; 25; 8.33; 2.78; 0.926; 0.309; 0.103; 0.034; 0.011 nM; and incubated for 72 h (+/−2 h).

Cell viability was determined on deposition of the cells (D-2), before the addition of the ADCs tested (D0) and 72 h after the addition of the compounds tested (D3), by measuring the amount of cellular ATP using the CellTiter-Glo® Luminescent Cell Viability Assay kit (Promega) according to the supplier's recommendations for use. Luciferase activity was measured on a luminometer (PerkinElmer® EnVision™).

Each concentration of compound was carried out in triplicate and two independent experiments were performed.

Results

The results, presented in FIG. 7, show a cytotoxic effect of McSAF-pyridine which is higher compared to T-DM1 and dependent on the presence of HER2.

### Example 5: Stability of the Bioconjugation

Plasma stability : quantification by LC-MS/MS of the amount of MMAE released after incubation in human plasma at 37° C. Comparison between McSAF-pyridine and an ADC produced with a maleimide attachment head (a single bond)=McSAF-maleimidel of the formula:

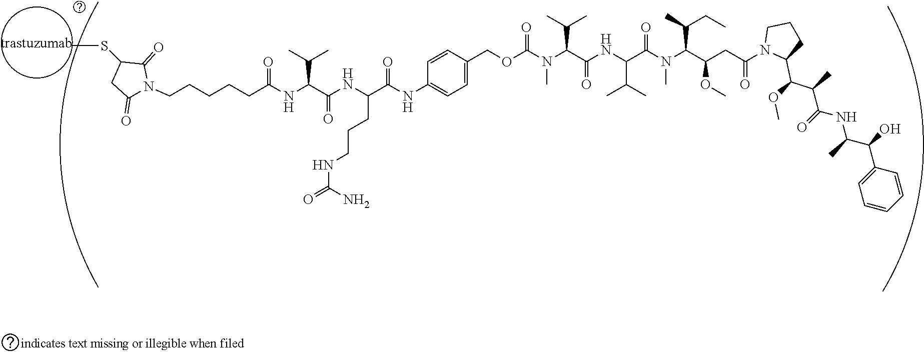

Materials and method

MMAE calibration curve

25 μL of RP424 (H₂O+0.1% FA) were added to 25 μL of samples (MMAE-ds standard or MMAE concentration range) and then the mixture was agitated. 75 μL of a standard solution “MMAE-d8” at 0.04 μg/mL were added before agitating for 30 seconds. The mixture was centrifuged at 20 000 g at 4° C. for 10 minutes.

The supernatant was removed and transferred to polypropylene flasks. A fresh centrifugation was carried out at 2500 g at 4° C. for 5 min before injection in LC-MS/MS. The samples were injected onto an Acquity BEH UPLC C18, 50×2.1 mm, 1.7 μm, column connected to an LC-20AD and LC-20ADXR system (Shimadzu) coupled to a Shimadzu mass detector (8060) with an ESI+ source.

Elution was performed with a gradient from 75% buffer D (10 mM ammonium acetate) in 25% buffer E (acetonitrile) to 5% buffer D in 95% buffer E over 5 minutes followed by an increase to 75% buffer D in 25% buffer E over 5.1 minutes. The results were processed with the Labsolution 6.60 software.

Human plasma incubation

The samples were incubated in sterile human EDTA-2K plasma (BiolVT) at an initial concentration of 100 μg/mL. 3 samples were collected just after having agitated the mixture (T0), then after 6 h, 12 h, 24 h, 48 h and 96 h of incubation at 37° C. The samples were stored at −80° C. prior to LC-MS/MS analysis thereof as described above.

Results

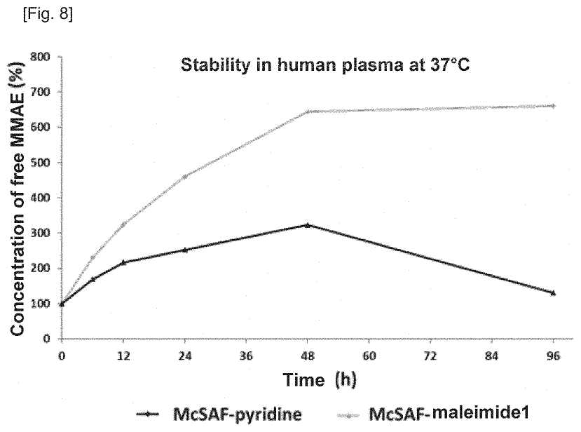

The results, presented in FIG. 8, show that McSAF-pyridine releases less MMAE into the plasma compared to McSAF-maleimide1. McSAF-pyridine is therefore more stable than McSAF-maleimidel under physiological conditions.

Stability at 37° C. in the presence or absence of HSA (human serum albumin)

Materials and method

The concentration of McSAF-pyridine and of McSAF-maleimide1, in a PBS buffer (1 mM monobasic sodium phosphate, 3 mM dibasic sodium phosphate, 155 mM sodium chloride, 1 mM sodium azide, pH 7.4), was adjusted to 2 mg/mL. Twelve (12) 20 μL samples of McSAF-pyridine and of McSAF-maleimidel were placed in twelve polypropylene flasks (Fisher Scientific, 0.6 mL) and then 20 μL of PBS buffer (1 mM monobasic sodium phosphate, 3 mM dibasic sodium phosphate, 155 mM sodium chloride, 1 mM sodium azide, pH 7.4) or 20 μL of a 20 mg/mL solution of HSA in PBS buffer (1 mM monobasic sodium phosphate, 3 mM dibasic sodium phosphate, 155 mM sodium chloride, 1 mM sodium azide, pH 7.4) were added to each flask. After agitation, each of the 12 flasks is centrifuged for 30 seconds at 5000 g before incubation at 37° C. in an incubator (VWR INCU-line IL23). The flasks were removed three by three from the incubator and stored at −80° C. after 1 minute (T0), 24 h, 48 h and 120 h.

After dilution by a factor of 2 with PBS buffer (1 mM monobasic sodium phosphate, 3 mM dibasic sodium phosphate, 155 mM sodium chloride, 1 mM sodium azide, pH 7.4), the contents of each of the 12 flasks are filtered through 0.22 μm and analyzed by HIC. For McSAF-pyridine, 50 μg of products were injected onto an MAbPac HIC-Butyl, 5 μm, 4.6×100 mm, column (ThermoScientific), connected to a Waters Alliance HPLC system (e2695) equipped with a PDA (e2998) set for detection at 280 nm. The samples were eluted at a flow rate of 1 mL/min with a gradient from 100% buffer A (1.5 M ammonium sulfate, 50 mM monobasic sodium phosphate, 5% isopropanol (v/v), pH 7.0) to 100% buffer B (50 mM monobasic sodium phosphate, 20% isopropanol (v/v), pH 7.0) in 50 minutes. The temperature was maintained at 25° C. throughout the separation.

For McSAF-maleimide1, 50 pg of products were injected onto a TSKgel Butyl NPR, 2.5 μm, 4.6×100 mm, column (Tosoh), connected to a Waters Alliance HPLC system (e2695) equipped with a PDA (e2998) set for detection at 280 nm. The samples were eluted at a flow rate of 0.6 mL/min with a gradient from 100% buffer A (1.5 M ammonium sulfate, 50 mM monobasic sodium phosphate, pH 7.0) to 20% buffer B (50 mM monobasic sodium phosphate, 20% isopropanol (v/v), pH 7.0) in 60 minutes and then this gradient was maintained for 6 min. The temperature was maintained at 30° C. throughout the separation.

Results

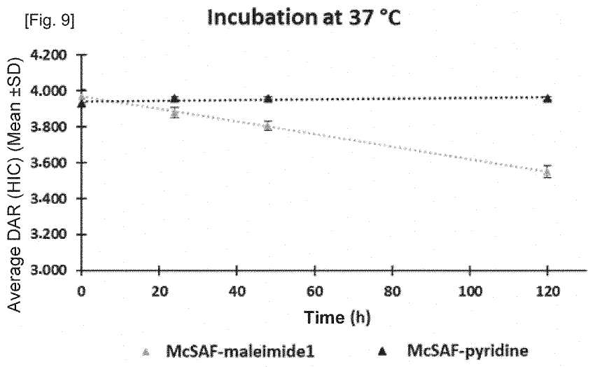

The monitoring of the change of the average DAR by the HIC method after incubation at 37° C. is presented in FIG. 9. The results show perfect stability at 37° C. for McSAF-pyridine, which is not the case for McSAF-maleimide1. This is explained by the increased stability of the antibody-drug conjugate according to the invention.

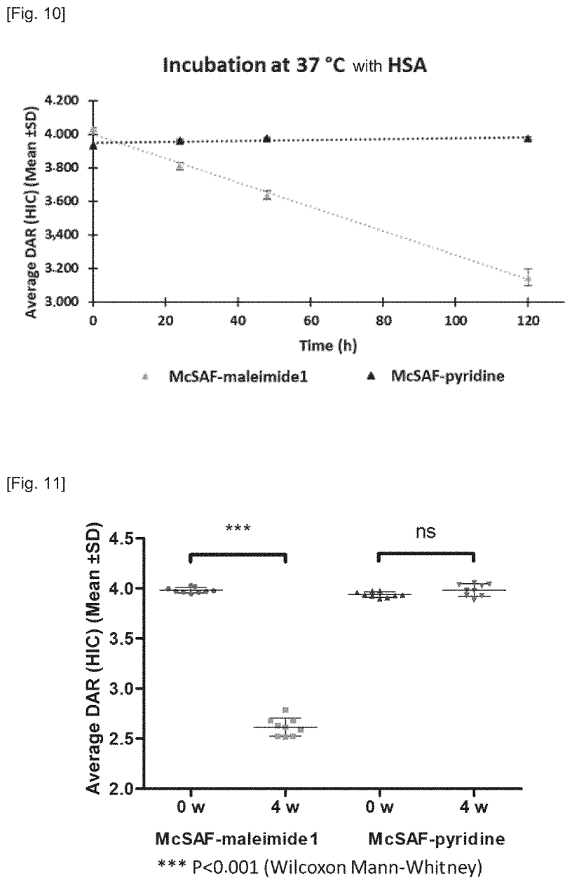

The monitoring of the change of the average DAR by the HIC method after incubation with an excess of HSA at 37° C. is presented in FIG. 10. The results show perfect stability in the presence of HSA for McSAF-pyridine, which is not the case for McSAF-maleimide1. This is explained by the increased stability of the antibody-drug conjugate according to the invention.

Stability at 40° C.

Materials and method

The concentration of McSAF-pyridine and McSAF-maleimide1, in a PBS buffer (1 mM monobasic sodium phosphate, 3 mM dibasic sodium phosphate, 155 mM sodium chloride, pH 7.4), was adjusted to 1 mg/mL. Six (6) 150 pL samples of McSAF-pyridine and of McSAF-maleimidel were placed in six polypropylene flasks (Eppendorf Protein LoBind, 0.5 mL). After agitation, the samples were incubated in an incubator (VWR INCU-line IL23) at 40° C. The flasks were removed three by three from the incubator, centrifuged for 2 minutes at 5000 g and stored at −80° C. after 1 minute and 4 weeks.

The contents of each of the 6 flasks were filtered through 0.22 μm and analyzed by HIC and SEC.

HIC

The McSAF-pyridine ADC was analyzed by carrying out the protocol described in example 3A.

For McSAF-maleimide1, 50 ═g of products were injected onto a TSKgel Butyl NPR, 2.5 μm, 4.6×100 mm, column (Tosoh), connected to a Waters Alliance HPLC system (e2695) equipped with a PDA (e2998) set for detection at 280 nm. The samples were eluted at a flow rate of 0.6 mL/min with a gradient from 100% buffer A (1.5 M ammonium sulfate, 50 mM monobasic sodium phosphate, pH 7.0) to 80% buffer B (50 mM monobasic sodium phosphate, 20% isopropanol (v/v), pH 7.0) in buffer A in 45 minutes and then this gradient was maintained for 6 min. The temperature was maintained at 30° C. throughout the separation.

SEC

50 μg of products were injected onto an AdvanceBio SEC, 2.7 pm, 7.8×300 mm, column (Agilent Technologies), connected to a Waters Alliance HPLC system (e2695) equipped with a PDA (2998) set for detection at 280 nm. The ADCs were eluted at a flow rate of 1 mL/min with an isocratic buffer C (1 mM monobasic sodium phosphate, 155 mM sodium chloride, 3 mM dibasic sodium phosphate, 3 mM sodium azide, pH 7.4) in 24 minutes. The temperature was maintained at 25° C. throughout the separation.

Results

The monitoring of the change of the average DAR by the HIC method after incubation at 40° C. for 28 days is presented in FIG. 11 (N=3). The results show that the average DAR of McSAF-maleimidel varies from 4.00 (at t0) to 2.61 (at t28), attesting to a lack of stability under the stressful conditions simulated, while that of McSAF-pyridine varies little (3.93 (at t0) to 3.98 (at t28)), demonstrating its improved stability compared to McSAF-maleimidel using maleimide technology.

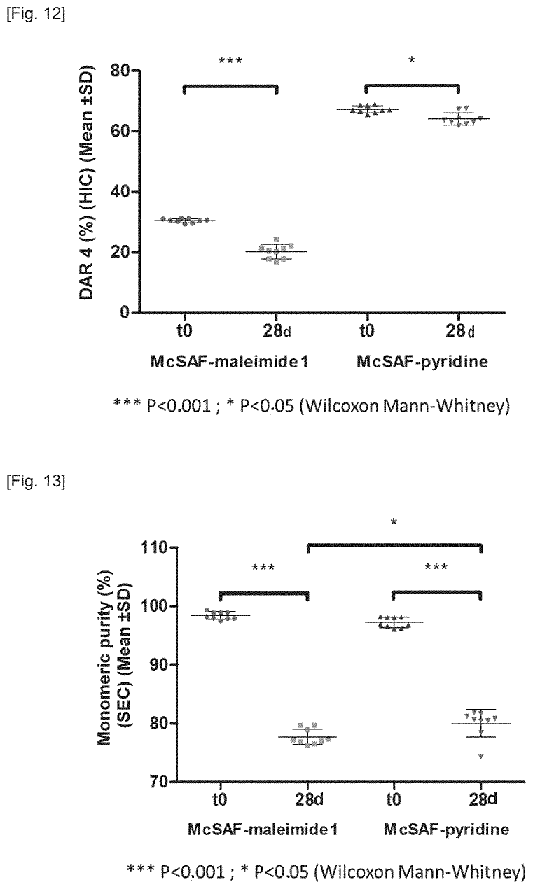

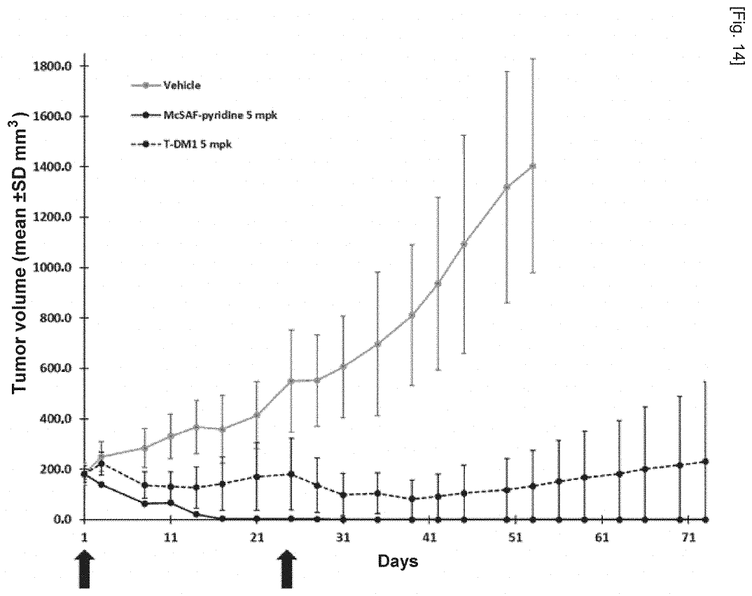

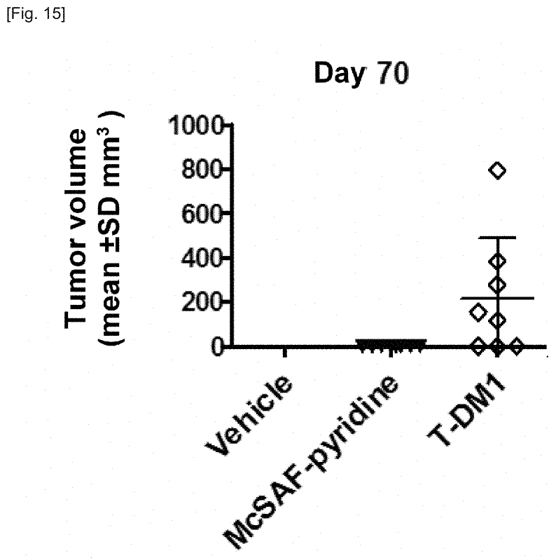

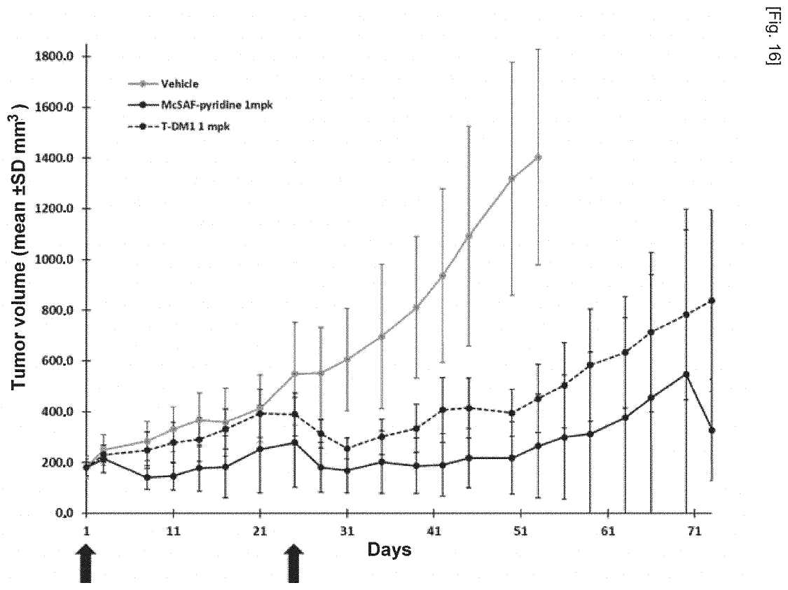

The monitoring of the change in the DAR4 percentage by the HIC method after incubation at 40° C. for 28 days is presented in FIG. 12 (N=3). The results show that the DAR4 percentage decreases over the course of time for McSAF-maleimidel (30% at t0 against 20% at t28) while that of McSAF-pyridine varies little (67% at t0 against 63% at t28). This demonstrates the increased stability and better preservation of the DAR4 percentage of McSAF-pyridine compared to McSAF-maleimidel using maleimide technology.

The monitoring of the change in the monomer percentage by the SEC method after incubation at 40° C. for 28 days is presented in FIG. 13 (N=3). The results show that the monomer percentage decreases in the same way for McSAF-maleimidel (77% at t28) and for McSAF-pyridine (80% at t28).

### Example 6: In vivo evaluation of the antibody-drug conjugate (McSAF-pyridine)

Materials and method

All experiments were carried out in an environment complying with the recommendations of the French authorities (Authorization No. B 21 231 011 EA) and of FELASA. The survival study was performed on a BT-474 xenograft model. A suspension of tumor cells was implanted subcutaneously in immunosuppressed BALB/c nude mice (Charles River) 24 to 72 h after complete irradiation with a γ source (2 Gy, 60Co, BioMep, Bretenieres, France). After the tumor graft had taken, the mice were randomized into groups (N=8) once the mean volume had reached 150-200 mm³. The ADCs (McSAF-pyridine or the T-DM1 reference) were administered on days 1 and 26 by the intravenous route (IV, bolus) into the caudal vein of the mice at an amount of 1 or 5 mg/kg. The tumor volume as a function of time was calculated twice a week using the following formula: (Length x thickness²)/2. The animals were euthanized when the tumor volumes reached 10% of their weight, or approximately 2000 mm³.

Results

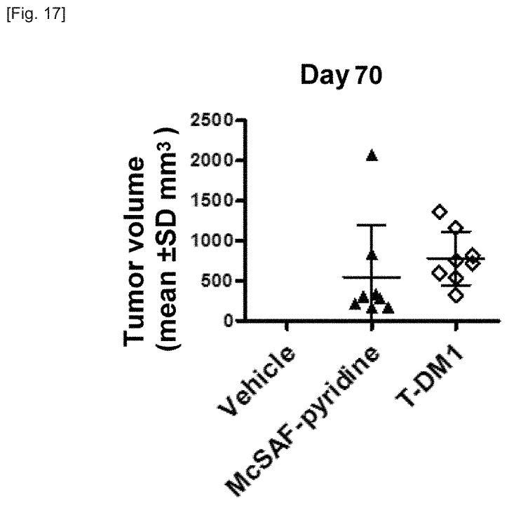

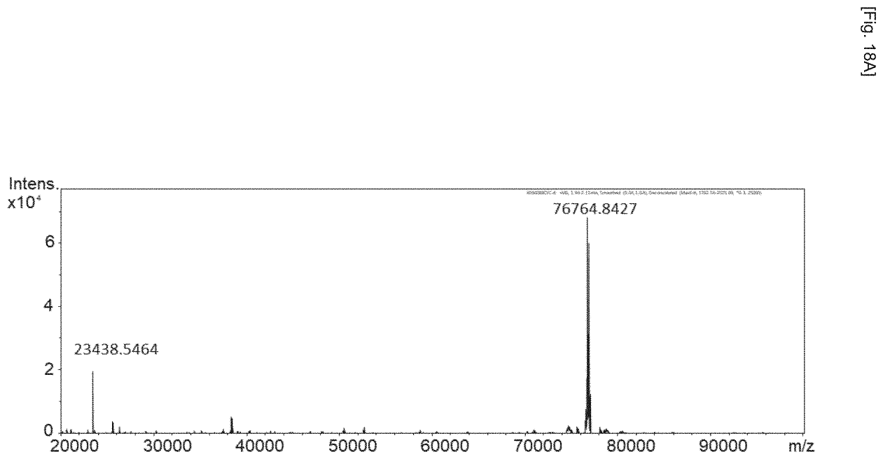

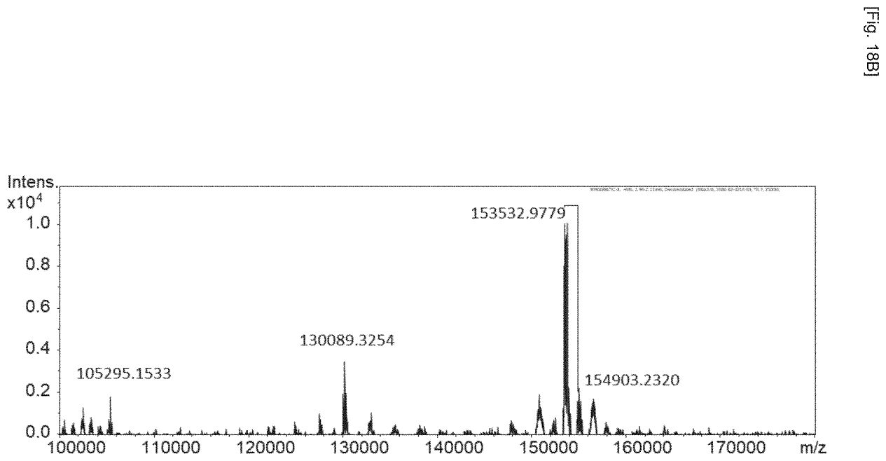

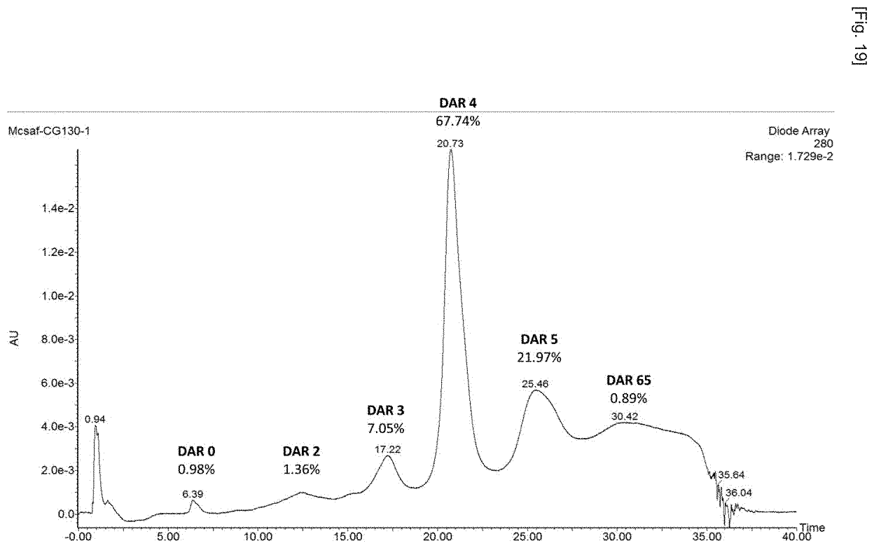

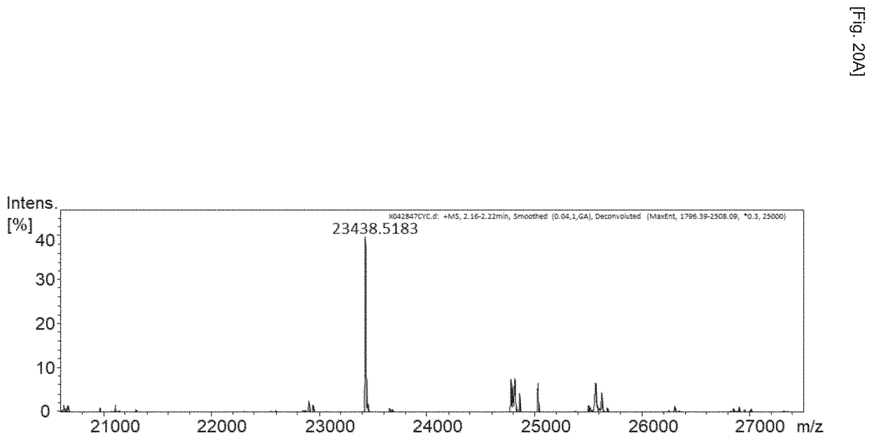

The results of administering a 5 mg/kg dose are presented in FIG. 14 and FIG. 15. The results of administering a 1 mg/kg dose are presented in FIG. 16 and FIG. 17. The results show that at both doses, 1 and 5 mg/kg, McSAF-pyridine is more effective than T-DM1. At 5 mg/kg of McSAF-pyridine, complete and lasting tumor regression was observed with 8 mice cured out of 8 mice treated.

A complementary immunohistochemistry (IHC) analysis on mice, at the end of the study, was performed. It made it possible to confirm the complete eradication of the HER2+ cells in the zone corresponding to the xenograft for the McSAF-pyridine ADC, for all the mice treated with 5 mg/kg, thus confirming complete tumor regression.

Sequence listing

| Sequence number | Sequence type | Amino acid sequence |
|---|---|---|
| SEQ ID NO: 1 | Trastuzumab light | DIQMTQSPSSLSASVGDRVTITCRASQ |
| | chain | DVNTAVAWYQQKPGKAPKLLIYSASFL |
| | | YSGVPSRFSGSRSGTDFTLTISSLQPE |
| | | DFATYYCQQHYTTPPTFGQGTKVEIKR |
| | | TVAAPSVFIFPPSDEQLKSGTASVVCLL |
| | | NNFYPREAKVQWKVDNALQSGNSQES |
| | | VTEQDSKDSTYSLSSTLTLSKADYEKH |
| | | KVYACEVTHQGLSSPVTKSFNRGEC |
| SEQ ID NO: 2 | Trastuzumab heavy | EVQLVESGGGLVQPGGSLRLSCAASG |
| | chain | FNIKDTYIHWVRQAPGKGLEWVARIYP |
| | | TNGYTRYADSVKGRFTISADTSKNTAY |
| | | LQMNSLRAEDTAVYYCSRWGGDGFYA |
| | | MDYWGQGTLVTVSSASTKGPSVFPLA |
| | | PSSKSTSGGTAALGCLVKDYFPEPVTV |
| | | SWNSGALTSGVHTFPAVLQSSGLYSLS |
| | | SVVTVPSSSLGTQTYICNVNHKPSNTK |
| | | VDKKVEPKSCDKTHTCPPCPAPELLGG |
| | | PSVFLFPPKPKDTLMISRTPEVTCVVVD |
| | | VSHEDPEVKFNWYVDGVEVHNAKTKP |
| | | REEQYNSTYRVVSVLTVLHQDWLNGK |
| | | EYKCKVSNKALPAPIEKTISKAKGQPRE |
| | | PQVYTLPPSREEMTKNQVSLTCLVKGF |
| | | YPSDIAVEWESNGQPENNYKTTPPVLD |
| | | SDGSFFLYSKLTVDKSRWQQGNVFSC |
| | | SVMHEALHNHYTQKSLSLSPGK |

References cited in the format “[reference number]”:
1. Fekete, S. et al., J. Pharm. Biomed. Anal., 130, 3-18, 2016
2. Barran, P. et al., EuPA Open Proteomics, 11, 23-27, 2016
3. Goyon, A et al., J. Chromatogr. B, 1065-1066, 35-43, 2017

1. A cytotoxic conjugate of formula (I) below: 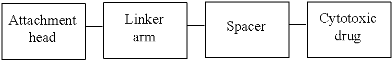
in which:
the attachment head is represented by either one of the two formulae below: 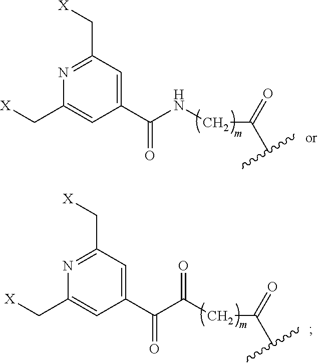
the linker arm is a cleavable linker arm chosen from the formulae below: 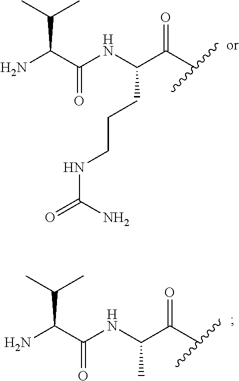 [SMILES: *C(=O)[C@H](CCCNC(N)=O)NC(=O)[C@@H](N)C(C)C ; *C(=O)[C@H](C)NC(=O)[C@@H](N)C(C)C]
the spacer is represented by the formula below: 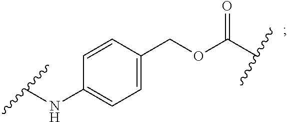 [SMILES: *Nc1ccc(COC(*)=O)cc1]
X is Br, Cl, I or F;
m is an integer ranging from 1 to 10.

2. The cytotoxic conjugate as claimed in of claim 1, wherein X is Br and m is equal to 4 or 5.

3. The cytotoxic conjugate of claim 1, wherein the cytotoxic drug is chosen from methotrexate, IMiDs, duocarmycin, combretastatin, calicheamicin, monomethyl auristatin E (MMAE), monomethyl auristatin F (MMAF), maytansine, DM1, DM4, SN38, amanitin, pyrrolobenzodiazepine, pyrrolobenzodiazepine dimer, pyrrolopyridodiazepine, pyrrolopyridodiazepine dimer, a histone deacetylase inhibitor, a tyrosine kinase inhibitor, and ricin, preferably MMAE.

4. The cytotoxic conjugate of claim 1, of formula (Ia) below:

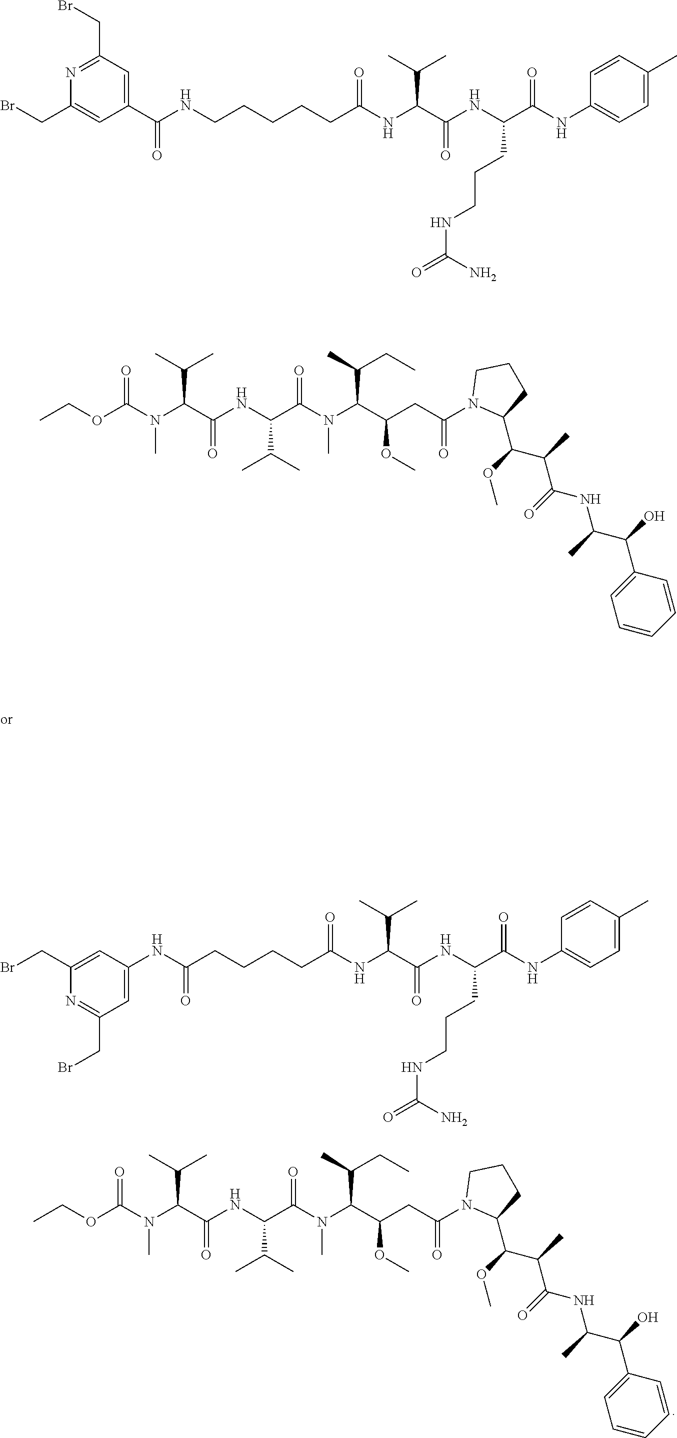

5. An antibody-drug conjugate of formula (II) below: 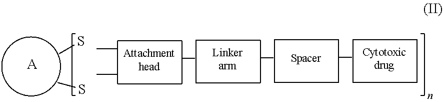
which:
A is an anti-HER2 antibody or antibody fragment;
the attachment head is represented by either one of the two formulae below: 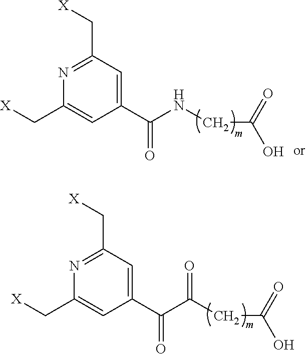
the linker arm is a cleavable linker arm represented by the formula below: 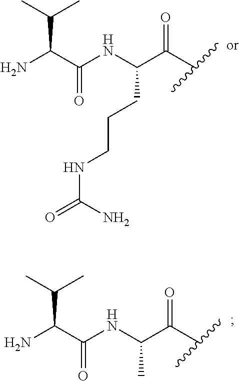 [SMILES: *C(=O)[C@H](CCCNC(N)=O)NC(=O)[C@@H](N)C(C)C ; *C(=O)[C@H](C)NC(=O)[C@@H](N)C(C)C]
the spacer is represented by the formula below: 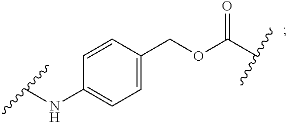 [SMILES: *Nc1ccc(COC(*)=O)cc1]
X is Br, Cl, I or F;
m is an integer ranging from 1 to 10;
n is an integer ranging from 1 to 4.

6. The antibody-drug conjugate of claim 5, wherein X is Br and m is equal to 4 or 5.

7. The antibody-drug conjugate of claim 5, wherein the cytotoxic drug is chosen from methotrexate, IMiDs, duocarmycin, combretastatin, calicheamicin, monomethyl auristatin E (MMAE), monomethyl auristatin F (MMAF), maytansine, DM1, DM4, SN38, amanitin, pyrrolobenzodiazepine, pyrrolobenzodiazepine dimer, pyrrolopyridodiazepine, pyrrolopyridodiazepine dimer, a histone deacetylase inhibitor, a tyrosine kinase inhibitor, and ricin, preferably MMAE.

8. The antibody-drug conjugate of claim 5, wherein A is trastuzumab.

9. The antibody-drug conjugate of claim 5, of formula (IIa) below:

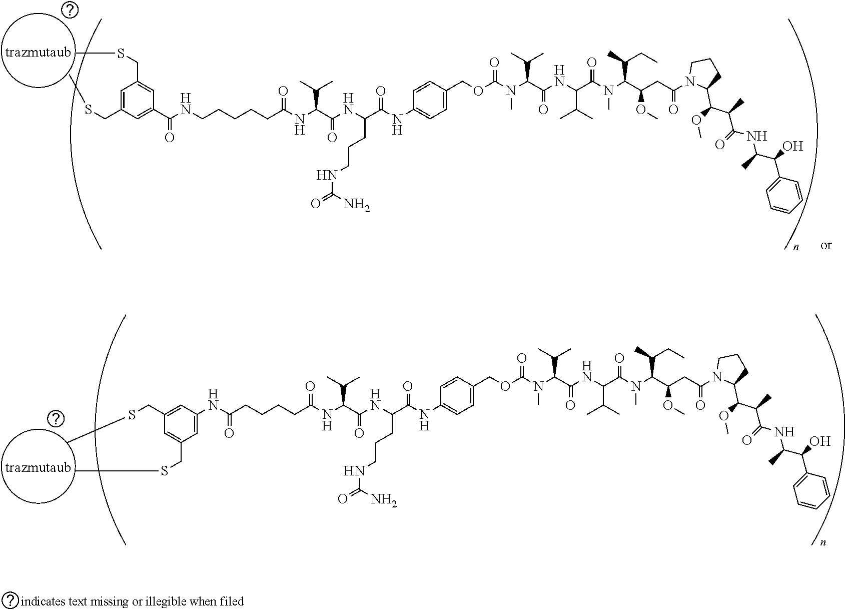

10. A composition comprising one or more antibody-drug conjugate(s) of claim 5.

11. The composition of claim 10, wherein at least 50%, preferably at least 65%, of the antibody-drug conjugates have an n equal to 4.

12. The composition of claim 10, wherein A is an antibody and the average Drug-to-Antibody Ratio (average DAR) is between 3.5 and 4.5, preferably between 3.8 and 4.2, for example equal to 4.0 ±0.2.

13. The composition as claimed in of claim 10, further comprising paclitaxel, docetaxel, doxorubicin, cyclophosphamide, an aromatase inhibitor such as anastrozole and/or an antibody used in anti-cancer immunotherapy such as an anti-PD1 antibody.

14. The antibody-drug conjugate of claim 4, for use as a medicament.

15. The antibody-drug conjugate of claim 4, for use in the treatment of an HER2+ cancer, preferably HER2+ breast cancer.

16. A process for preparing a cytotoxic conjugate of claim 1, comprising a step which consists in coupling an attachment head of formula: 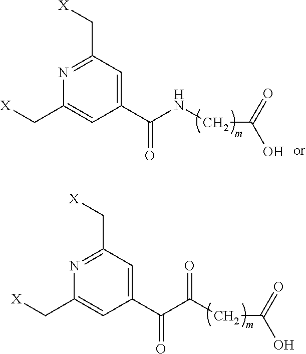
with a compound of formula 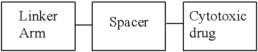
in which:
the linker arm is a cleavable linker arm chosen from the formulae below: 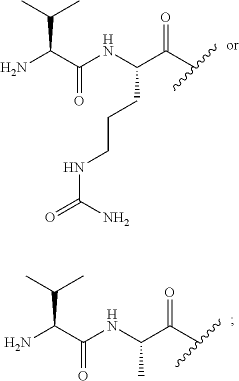 [SMILES: *C(=O)[C@H](CCCNC(N)=O)NC(=O)[C@@H](N)C(C)C ; *C(=O)[C@H](C)NC(=O)[C@@H](N)C(C)C]
the spacer is represented by the formula below: 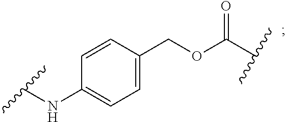 [SMILES: *Nc1ccc(COC(*)=O)cc1]
X is Br, Cl, I or F;
m is an integer ranging from 1 to 10, and preferably equal to 4 or 5.

17. A process for preparing an antibody-drug conjugate of claim 5, comprising the following steps:
(i) preparing a cytotoxic conjugate according to the process of claim 16, and
(ii) reacting the cytotoxic conjugate obtained in step (i) with an anti-HER2 antibody or an anti-HER2 antibody fragment.
